(19) United States
(12) Patent Application Publication
(10) Pub. No.: US 2022/0025458 A1
(43) Pub. Date: Jan. 27, 2022

(54) METHOD FOR PROVIDING AN IDENTIFIER FOR A PRODUCT

(71) Applicant: ORVINUM AG, Magden (CH)

(72) Inventors: Jurg RICHTER, Magden (CH); Markus EHRAT, Magden (CH); Anna WESTON, Basel (CH); Eric KUBLER, Basel (CH)

(21) Appl. No.: 17/298,471

(22) PCT Filed: Nov. 29, 2019
(86) PCT No.: PCT/EP2019/083165

§ 371 Date: May 28, 2021

(30) Foreign Application Priority Data
Nov. 30, 2018 (EP) 18209576.0

(51) Int. Cl. C12Q 1/6876

(52) U.S. Cl. CPC C12Q 1/6876

(57) ABSTRACT

The present invention relates to a method for providing an identifier of a product which allows to specifically identify a product or test its authenticity, e.g. in the form of a digital finger print. Thereby, the product is food stuff, and in particular processed food stuff, for instance wine. Said identifier can be obtained using sequencing and/or microarray analysis of nucleic acid sequences from the microbiome and/or macrobiome of said food product, in particular wine.

### REFERENCE TO SEQUENCE LISTING SUBMITTED VIA EFS-WEB

This application includes an electronically submitted sequence listing in .txt format. The .txt file contains a sequence listing entitled “VOS-101US_ST25.txt.txt” created on May 28, 2021 and is 10,446 bytes in size. The sequence listing contained in this .txt file is part of the specification and is hereby incorporated by reference herein in its entirety.

### FIELD OF THE INVENTION

The present invention relates to product identification/authentication. In particular, the present invention relates to a method for evaluating the authenticity of a food product, e.g. by use of a digital finger print. The product can be food stuff, and in particular processed food stuff, for instance wine.

### DESCRIPTION OF THE RELATED ART

Methods for specifically identifying products such as food stuff are known. Often, they are used for differentiating falsification of such products. A simple method is known for instance from CN101665825, which adds DNA of a known sequence to spirit, which is used later to discriminate original spirit from counterfeit products. However, addition of any kind of material to a food simply for identification purpose, in particular if it is related to DNA, may not be accepted by consumers.

Therefore, further technologies have been developed which allow the identification of specific products. In a way, they all relate to the identification and verification of markers which are specific and unmistakable for the product.

Nucleic Acid Sequence Detection

Nucleic acid sequences are very specific for certain organisms, therefore the detection of species specific sequences in food stuff allows for a clear identification of the ingredients and potential contaminations. Shahrooz et. al. (Food Control 68 (2016) 379-390) describe in their review the identification of different types of meat species by hybridizing the ssDNA sequence to a complementary sequence on e.g. microarrays or by the application of PCR amplification methods.

Pathogen Detection

Simultaneous detection of the presence of pathogenic microorganism is another topic of interest in food stuff analysis. Zhaohuan Zhang et. al describe in their paper (Food Control 51, 31-36 (2015)) the development of a multiplex real-time PCR method for the simultaneous detection of three pathogens in seafood.

Ingredient Identification

The analysis of specific DNA sequences also allows determining the microbial composition of food raw materials which can have an impact on the quality of the processed food. Cécile Philippea et. al describe in their paper “A survey of oesophagus during wine making reveals a novel group with unusual genomic characteristics” Journal of Food Microbiology 257, 138-147 (2017) that it is generally accepted by the scientific community that the quality and identity of wines will benefit from a better knowledge of the bacterial, yeast and fungal communities associated with soils, grapes and products during fermentation and that the unveiling of the microbiome and its dynamics during wine making is expected to help in the identification of the ecological factors and farming systems that explain the biodiversity and guide practices to avoid wine quality depreciation.

Raw Material Composition and Impact on Food Processing

Furthermore N. A. Bokulich et. al describe in their paper “Associations among Wine Grape Microbiome, Metabolome, and Fermentation Behavior Suggest Microbial Contribution to Regional Wine Characteristics” mBio, American society for microbiology, May/June 2016 Volume 7 Issue 3, 631-16, how microbial dispersion pattern contributes to the regional wine characteristics and that microbial activity is an integral part of wine production and that both grape microbiota and wine metabolite profiles distinguish viticultural area designations and individual vineyards within Napa and Sonoma Counties, California. Associations among wine microbiota and fermentation characteristics suggest new links between microbiota, fermentation performance, and wine properties.

Rob Knight et. al. describe in US 2015/0284810 A1 the use of microbiome information for the control of industrial processes. Particularly they describe how the art of industrial monitoring, automation and control so far has largely ignored the microbial and genetic information that is present in, or associated with an industrial operation. Analyzing microbial materials from locations associated with industrial operations allows to better characterize and control industrial processes.

Authentication and Identification of Samples

In WO 99/46405 unique DNA sequences are provided which are useful in identifying different fermentation-related microorganisms. These unique DNA sequences can be used to provide oligonucleotide primers in PCR based analysis for the identification of fermentation related microorganisms. The DNA sequences described in WO 99/46405 include the internal transcribed spacer of the ribosomal RNA gene regions of particular fermentation related microorganisms, as well as oligonucleotide primers which are derived from these regions which are capable of identifying the particular microorganism.

Valentina Catalano et al. (Journal of Agricultural and Food Chemistry, 64(37), p. 6969-6984) discloses the development of an authentication system for the traceability of wine. This approach is focused on the analysis of the macrobiome, i.e. plant DNA, during different steps of winemaking. In particular, the presence of different nuclear and chloroplast microsatellite markers was analyzed by PCR using specific primers for these markers and subsequent analysis of the fragments by electrophoresis. Unlike the method of the present invention, the method disclosed by Catalano et al. does not allow analyzing the macrobiome and the microbiome of the wine simultaneously, and is thus unlikely to discriminate between wines with only subtle differences, for example, wines from the same producer but from different vintages.

Montet et al. (Aspects of Applied Biology, 87, XP055551730) discloses a method for analyzing the variation in microbial communities in fish and fruit between samples from different geographical origins by PCR-DGGE (polymerase chain reaction—denaturing gradient gel electrophoresis). In particular, the 16S rRNA gene of bacteria and the 26S rRNA gene of yeasts was analyzed in this approach. By comparing the band patterns obtained by PCR-DGGE with known samples it was possible to draw conclusions about the origin of a sample. However, the analysis by PCR-DGGE does not provide sequence information and thus does not allow reliable identification of microbial species in the sample. Further, the approach by Montet et al. does not include analysis of the macrobiome of the sample.

Savazzini and Martinelli (Analytica Chimica Acta, 563(1-2), p. 274-282) disclose a method for DNA analysis in wine by real-time PCR. In this approach, DNA derived from the macrobiome and the microbiome of the wine was analyzed. Analysis of the macrobiome was achieved by designing primers for the detection of specific microsatellites in the plant DNA. However, analysis of the microbiome only extended to detecting the presence of DNA from the yeast Saccharomyces cerevisiae. Thus, the method does not allow a detailed analysis of the wine microbiome as the method of the present invention. Further, the method requires the design of specific probes for each microbial species that is to be analyzed which is not feasible in view of the large space of microbial species. Further, this approach requires previous knowledge about the composition of the sample, e.g. the species that are expected in the sample.

Arcuri et al. (Food Control, 30(1), p. 1-6) discloses a method for determining the origin of cheese by analyzing bacterial 16S rDNA by PCR-DGGE. As in the case of the approach disclosed by Montet et al., only the band pattern obtained by PCR-DGGE with different samples was compared in order to draw conclusions on the origin of the cheese samples. Thus, no detailed information about the composition of the microbial community in a sample was obtained with this method. It has to be noted that in this approach single bands obtained by PCR-DGGE were excised and sequenced by Sanger sequencing. However, analyzing only single bands does not provide information of the microbial composition in the entire sample and was merely performed to identify previously unknown bacteria that are involved in the aging of the cheese.

### OBJECTIVE OF THE INVENTION

Accordingly, there is a need in the art for a method that allows the authentication of food products by analyzing DNA in the product that has been derived from components of the macrobiome and/or the microbiome.

It is desirable to provide a method for the authentication and/or characterization of wine and/or other foodstuff capable to perform discriminations in great depth and detail level, e.g. to discriminate wine from different producers of a given region or of different vintages and/or to provide information on the quality of foodstuff regarding e.g. the main ingredients and potential contaminations.

It is also desirable to provide a method that is robust against the impact of nucleic acid amplification inhibitors present in the sample, partial degradation of nucleic acids, e.g. during storage of a sample or partial depurination of nucleic acids.

Furthermore, it is desirable to provide an identifier such as a digital, in particular binary, nucleic acid based code for a sample allowing the specific authentication and/or falsification or authenticity of a sample.

Also, it is desirable to provide a method that allows to overcome or even take advantage of age-related changes of analytes investigated in a test sample, in particular a test sample of foodstuff, in particular wine. The average skilled person will be aware that in particular wines, more specifically very expensive wines of high quality, are frequently stored for a very long time, such as several decades or even centuries. Whereas decay of some analytes dependent on the duration of storage allows to derive age dependent parameters, e.g. the production year, other analytes are more stable over the shelf life period, i.e. they are not undergoing changes to an extent that has a significant impact on the test results. These seem to be easily suitable for the determination of the sample origin. However, disregarding less stable analytes sometimes is disadvantageous. Some substances decay over the course of time. As long as the concentration of such substances is still measurable, it could be used as an indicator of age, e.g. in order to determine a production year. In particular, concentration ratios of decaying vs. stable molecule concentrations could be considered.

It may be desirable to provide the capability to determine nucleic acids of microorganisms, fungi, yeasts, bacteria, viruses, phages, archaea, protists and/or plants in a sample to create an identifier code for each product, e.g. based on a multitude of nucleic acid sources. The identifier code may be a digital identifier code.

It may be desirable to provide the capability to determine cellular components such as peptides and proteins of microorganisms, fungi, yeasts, bacteria, viruses, phages, archaea, protists and/or plants in a sample to create an identifier code for each product, e.g. based on a multitude of nucleic acid sources. The identifier code may be a digital identifier code

It may be desirable to provide a device for performing the method according to the invention, and a kit enabling performing the inventive method.

The technical problem is solved by the embodiments provided herein, and as characterized in the independent claims. Some advantageous embodiments of the invention are specified in dependent claims, other advantageous embodiments will be found in the description.

If not specified otherwise, all embodiments of the invention disclosed in the following can be combined with one another.

That is, the present invention relates to the following items:

1. A method for providing an identifier for a product, in particular for wine or for food stuff, in particular for a processed food stuff product, the product comprising a product specific ensemble of molecules from a set of different distinguishable molecules, the method comprising the steps of: a) obtaining a sample of the product; b) analyzing the sample in a manner using a set of molecules capable of recognizing and/or binding selected target molecules or parts thereof in generating a set of signals having strengths allowing determination of whether or not and/or to what extent molecules from the set of different distinguishable molecules are to be considered to constitute part of the specific ensemble of molecules in the sample, c) compiling an identifier having a plurality of elements in view of signals from the set of signals in a manner using a plurality of the signals in determining the plurality of elements.

2. The method for providing an identifier for a product according to item 1, wherein compiling the identifier having a plurality of elements in view of signals from the set of signals comprises comparing signal strengths of the set to thresholds to determine comparison results, the comparison results indicating whether or not and/or to what extent a respective of the different distinguishable molecule is to be considered present in the sample, a molecule from the set of different distinguishable molecules being considered to constitute part of the specific ensemble of molecules in the sample and/or to be present to an extent specific for the ensemble depending on whether a respective signal strength exceeds a specific threshold and/or remains below a specific threshold; and compiling the comparison results into the identifier.

3. The method for providing an identifier for a product according to item 2, wherein comparing signal strengths of the set to thresholds comprises comparing at least one of the signal strengths to both a specific lower threshold and a specific upper threshold and at least one molecule from the set of different distinguishable molecules is considered to constitute part of the specific ensemble of molecules in the sample only if the respective signal strength exceeds a specific lower threshold, but remains below a specific upper threshold; and/or wherein the identifier is compiled in view of whether for at least one specific signal strength a comparison against more than one threshold has indicated the respective molecule is to be considered to constitute part of the specific ensemble of molecules in the product and/or wherein the one or more specific threshold to which the identifier in view of signals from the set of signals is compared is determined in view of a confidence interval of the signal strength and/or in view of the kinetic behavior of at least one threshold and/or wherein the set of thresholds to which the set of signals strengths is compared is determined with a view on a set of signals strengths obtained for a comparable product known to be genuine.

4. The method of any one of the previous items, wherein identical or similar genuine products may have been stored under conditions prior to providing a sample that are different to conditions under which a candidate product has been stored prior to obtaining a sample, and wherein at least one of the molecules from the set of different distinguishable molecules is unstable to an extent such that evaluating the strength of the respective signal by comparing it to at least one given threshold obtained for a sample from a product stored according to first conditions is expected to yield a result different from comparing the strength of a corresponding signal to the threshold obtained for a sample from a product stored according to second conditions different from the first conditions, and wherein further the method comprises a step of adjusting the evaluation, in particular a threshold, prior to determining the identifier element, to the storage conditions of candidate product, the method preferably comprising a step of adjusting the threshold to storage conditions known or assumed to be likely, in particular to a period of storage of the candidate product.

5. The method for providing an identifier for a product according to one of the previous items, wherein compiling the identifier in view of signals from the set of signals having a plurality of elements comprises determining at least one ratio of signal strengths and evaluation of the ratio in view of at least one of one other ratio obtained for a different combination of signal strengths, or in view of an expected decay behavior of the molecules the signal strengths relate to.

6. A method according to one of the previous items, wherein the target molecules which the set of molecules is capable of recognizing and/or binding are molecules from the set of different distinguishable molecules and/or are derived from such molecules during analysis of the sample.

7. A method according to one of the previous items, wherein the product is a food stuff, and is in particular a processed product, and wherein the set of molecules capable of recognizing and/or binding selected target molecules comprises molecules capable of recognizing and/or binding as target molecules nucleic acid molecules or peptides, or small or large molecules in particular those comprised in members of the microbiome and/or macrobiome of the sample and/or derived therefrom during storage and/or analysis.

8. The method of one of the previous items, wherein the product is wine and the set of molecules capable of recognizing and/or binding selected target molecules comprises molecules capable of recognizing and/or binding as target molecules nucleic acid molecules or peptides or small or large molecules or large molecules comprised in members of the microbiome of the wine, in particular in the microbiome comprising fungi, yeasts, bacteria and/or phages and/or target molecules derived from members of the microbiome of the wine during storage and/or analysis and/or comprised in members of the macrobiome in particular comprising plants, in particularly vine, and/or target molecules derived from members of the macrobiome of the wine during storage and/or analysis.

9. The method according to one of the preceding items, wherein one or multiple sets of molecules capable of recognizing and/or binding selected target molecules that are used in step b) are specific for genera, preferably species, comprised in the macro- and/or microbiome comprised in the sample and/or are nucleic acid molecules, or antibodies or antibody-like polypeptides, or peptides.

10. The method according to one of the preceding items, wherein the set of molecules capable of recognizing and/or binding selected target molecules comprises at least one nucleic acid molecule and wherein step b) comprises the use of hybridization of at least one nucleic acid molecule to complementary sequences for DNA microarray assays, PCR amplification methods and/or sequencing, in particular next generation sequencing, in particular wherein said PCR amplification method is multiplex real-time PCR and/or wherein said at least one nucleic acid molecule targets the bacterial 16S rRNA genes and/or wherein the molecules capable of recognizing and/or binding selected target molecules comprise at least one antibody, antibody fragment or antibody-like polypeptide or aptamer and/or wherein step b) comprises the use of ELISA methods, and wherein in particular the ELISA method comprises use of a secondary antibody or antibody-like polypeptide for detection.

11. The method according to one of the preceding items, wherein compiling the identifier, in particular compiling in a method according to one of items 2 to 5, comprises generation of a binary matrix, preferably a binary matrix having N bits with N corresponding to or being larger than the number of distinguishable different molecules in the set of different distinguishable molecules.

12. A method of evaluating the authenticity of a candidate product comprising the steps of providing an identifier for the candidate product according to one of previous items, determining from a library of information relating to products known to be genuine one or more properties the identifier of the candidate product is expected to have to be authentic, comparing the one or more properties determined from the library to the respective one or more property of the identifier of the candidate product, judging that the candidate product should not be considered authentic if one or more properties of the identifier of the candidate product does not compare favorably to the one or more properties of the identifier of the genuine product, in particular wherein the candidate product is wine and the information relating to a product known to be genuine retrieved from the library is determined based on a labeling of the candidate product, in particular such that the product known to be genuine is the same wine from the same producer and the same vintage or is one or more of the same wine from the same producer but from a different vintage, in particular one or more vintage from a year close to the vintage of the candidate product with respect to time and/or growing conditions or one or more of the same wine from a different producer but the same region and preferably wherein the one or more property of the identifier is determined with a view on whether the product known to be genuine is the same wine from the same producer and the same vintage or the same wine from the same producer but from a different vintage or the same wine from a different producer but the same region, and particularly wherein the candidate product is a candidate wine for which no identical wine from the same producer and the same vintage is included in the library and wherein the one or more properties of the identifier of the candidate wine is expected to have is evaluated with a view on an importance of the one or more properties, in particular such that to one or more properties relating to members of the macrobiome of the wine, in particular comprising plants, in particularly vine, an importance higher than the importance of one or more properties relating to members of the microbiome of the wine, in particular in the microbiome comprising fungi, yeasts, bacteria and/or phages is assigned, and wherein preferably in judging the candidate product to be authentic, a weight is assigned to the properties dependent on their importance.

13. The method according to the preceding item, wherein rejecting the assumption of a candidate product being authentic is attempted in an iterative manner, comprising the steps of providing in a first iterative step a first part of the identifier information of the candidate product, attempting to falsify that the candidate product is authentic based on one or more properties of the first part of identifier information, providing a further part of the identifier information of the candidate product in case the candidate product cannot be falsified in a previous step, attempting to falsify that the candidate product is authentic based on the further information, in particular repeating the iteration until either the assumption of authenticity is falsified or identifier information relating to all molecules of the set of different distinguishable molecules has been evaluated.

14. A method of establishing a library of information relating to different genuine products and usable for authentication of candidate products, the genuine products comprising a plurality of distinguishable molecules in concentrations that for at least some of the genuine products and some of the molecules differ for the different genuine products, the method comprising the steps of: a) obtaining samples of the genuine products; b) analyzing the samples in a manner using a plurality of molecules capable of recognizing and/or binding selected target molecules in generating a plurality of signals having strengths allowing determination of whether or not and/or to what extent molecules from the set of different distinguishable molecules are to be considered to constitute part of the specific ensemble of molecules in the sample, in a respective of the samples, and storing information relating to a strength of the signals in a manner such that a comparison can be made between the information stored and an identifier obtained in a method according to one of the preceeding claims, wherein in particular the method of establishing a library of information preferably comprises establishing a plurality of thresholds of signal strengths such that a molecule from the set of different distinguishable molecules can be considered to be present in the sample depending on whether or not the respective signal strength exceeds the threshold and information relating to the established thresholds is stored in the library, and/or wherein in particular from the plurality of distinguishable molecules in the set of distinguishable molecules a the ensemble is selected such that genuine products may be discriminated from each other based on thresholds that allow for errors in adjusting to different storage conditions.

15. A kit comprising at least a container for a sample of a product obtained in a manner allowing determination of an identifier according to one of the preceding method items; and instructions to execute or have executed a method according to one of the preceding method claims, and/or comprising primers for the detection of components of the macrobiome and/or microbiome in a manner allowing determination of an identifier according to one of the preceding method claims; and/or comprising a fluidic array with one or more primer(s) to perform multiplexed PCR in a manner allowing determination of an identifier according to one of the preceding method claims; and/or comprising a microarray with one or more oligonucleotide(s) to perform hybridization assays in a manner allowing determination of an identifier according to one of the preceding method claims.

In general terms, the present invention provides a method for the identification and/or authentication of a product by correlating a set of specific (binding and/or recognizing) molecules with a set of target molecules contained in or derived from a sample of said product.

Accordingly, in one embodiment, the invention relates to a method for evaluating the authenticity of a food product, the method comprising the steps of: (a) obtaining a sample of the food product; (b) generating a plurality of signals based on the presence and/or the amount of two or more target molecules in the sample obtained in step (a), wherein the generation of the plurality of signals comprises a sequencing method and/or a microarray assay; (c) compiling an identifier having a plurality of elements based on the plurality of signals generated in step (b); (d) determining one or more properties the identifier of the food product is expected to have to be authentic; (e) comparing the one or more properties determined in step (d) for the food product to the respective one or more properties of an identifier of a product that is known to be authentic; and (f) evaluating the authenticity of the candidate product based on the comparison made in step (e).

That is, the method of the present invention may be used for the authentication of food products. Previous methods for the authentication of food products mainly rely on PCR-based methods which fail to provide a complete and detailed picture of the macrobiome and/or microbiome of the food product. For example, PCR-DGGE-based approaches only provide band-patterns that can be compared between samples, but do not provide any information about the organisms the target molecules in the samples have been derived from. Identifying these organisms in the sample would then require specific primers or probes for each organism which would, in turn, require previous knowledge about the target molecules that are expected to be comprised in a sample.

The method of the present invention allows a much more detailed analysis of target molecules in a sample, which allows a more detailed and reliable verification of the origin and the authenticity of a food product. To the surprise of the inventors, the sensitivity and accuracy of the method of the invention is high enough to even discriminate between wines from the same producer but from different vintages (See Example 1 and Table 3). This discrimination is possible, to a large extent, due to subtle variations in the microbiome of the wine which would be highly unlikely to be identified with the methods known in the art.

Within the present invention, it is envisioned that for the precise and reliable authentication of a food product at least two target molecules are analyzed. However, it has to be noted that in some instances and for certain food products analysis of a single target molecule may be sufficient for the authentication. Accordingly, the invention further encompasses embodiments in which an identifier comprises only a single element which corresponds to the analysis of a single target molecule.

The method of the present invention may be used for analyzing the authenticity of any kind of food product. Preferably, the method may be used for the authentication of processed food products, wherein the method of the invention may be used for the analysis of the macrobiome and the microbiome of the processed food product. In a more preferred embodiment, the method according to the invention may be used for the analysis of liquid food products. In particular, the method according to the invention may be used for the authentication of alcoholic beverages such as wine, whiskey or cognac. In a most preferred embodiment, the method of the invention is used for the authentication of wine. Alternatively, the method of the invention may be used for the authentication of oils, in particular olive oil.

The method comprises the step of obtaining a sample from the food product. A sample may be taken by any method known in the art. In case of a liquid, a sample may be obtained by collecting a defined volume of the liquid. When the food product is a homogenous, preferably liquid, food product, it may be sufficient to obtain a single sample. However, multiple samples may be taken to obtain a more reliable analysis. If the food product is a heterogeneous food product, it may be advisable to obtain multiple samples from different parts of the food product to obtain a reliable analysis.

For each sample an identifier is compiled based on the plurality of signals that are generated for the target molecules in the sample. The term “identifier,” as used herein, refers to a combination of symbols, names, numbers or any other means that can be used for identifying a sample. Within the present invention, an identifier comprises a plurality of elements, wherein each element may correspond to a target molecule in a sample. Each element of the identifier may comprise specific information about the presence and/or the amount of a corresponding target molecule in a sample. In certain embodiments, this information may be a yes/no decision if a target molecule is present in a sample or not. In other embodiments, this information may be if a target molecule is present in a sample at a specific concentration or within a specific range of concentrations. In other embodiments, this information may be if a target molecule is present in a sample at a concentration that is lower or higher than an internal and/or external standard. In other embodiments, this information may be if a target molecule is more, less or equally abundant than in another sample, such as a sample that has been obtained from a product that is known to be authentic. Within the present invention, not all elements of the identifier need to comprise the same type of information.

For determining the authenticity of a food product, i.e. a candidate product, the identifier that has been compiled for said food product is compared to an identifier that has been compiled for a product that is known to be authentic. The identifiers for the candidate product and the product that is known to be authentic may be generated simultaneously. That is, for example, the identifiers for both products may be compiled based on signals that have been generated in the same experiment, for example the same next-generation sequencing run. Alternatively, the identifier for the product that is known to be authentic may also be compiled at a previous time point compared to the identifier for the candidate product. That is, the identifier for the product that is known to be authentic may be part of a library of identifiers.

A library of identifiers may be generated by compiling identifiers for two or more products that are known to be authentic by applying the method of the present invention to each product, that is, by (a) obtaining a sample of the food product; (b) generating a plurality of signals based on the presence and/or the amount of two or more target molecules in the sample obtained in step (a), wherein the generation of the plurality of signals comprises a sequencing method and/or a microarray assay; and (c) compiling an identifier having a plurality of elements based on the plurality of signals generated in step (b).

A library of identifiers may comprise a multitude of identifiers for various food products. That is, the method of the invention may not only be used for authenticating a candidate product but also to identify an unknown food product, given that an identifier for an identical product is comprised in the library. However, even if no identifier for an identical product is comprised in the library, the method of the invention may be used to identify the known product with the highest similarity to the unknown product.

When comparing the identifier of a candidate product to the identifier of a product that is known to be authentic, it is preferred to compare only elements of the identifiers that correspond to the same target molecules. In addition, the comparison may be made on the whole set of elements that relate to the same target molecules or only to a subset of elements that are expected to be significant for the identification and/or authentication of a food product.

In particular, the present invention may relate to a method and processes for the identification and authentication of samples based on nucleic acid profiles. These nucleic acid profiles may be obtained from nucleic acid sequences of the sample's main components or may be relating to such main components or ingredients, as well as to the whole or part of the population of organisms that once were in contact with or still are present in the sample. The sample can be obtained in particular from foodstuff, in particular wine. Especially, the method may comprise steps of providing one or multiple sets of nucleic acid fragments specific to certain selected species in the sample such as plants, microorganisms, fungi, yeasts, bacteria, viruses, phages, archaea, protists, and to use said nucleic acid fragments in analyzing samples in a specific manner with or without prior amplification and to then create e.g. a specific digital pattern.

The present invention also encompasses a device designed for allowing to perform the inventive method, the flow device being designed to enable the isolation of nucleic acid fragments from the sample, performing an optional nucleic acid digestion and/or amplification as well as subsequent sequence determination or sequence specific detection and/or quantification. Furthermore the invention encompasses a kit enabling performing the inventive method.

According to one general aspect, a method for providing an identifier for a product, in particular for wine or for foodstuff, in particular for a processed foodstuff product, the product comprising a product specific ensemble of molecules, or target molecules, from a set of different distinguishable molecules, is suggested, the method comprising the steps of: a) obtaining a sample of the product; b) analyzing the sample in a manner, using a set of molecules capable of recognizing and/or binding selected target molecules and/or parts thereof in generating a set of signals having strengths allowing determination of whether or not and/or to what extent molecules from the set of different distinguishable molecules are to be considered to constitute part of the specific ensemble of molecules in the sample, c) compiling an identifier having a plurality of elements in view of signals from the set of signals in a manner using a plurality of the signals in determining the plurality of elements.

Note that the molecules of the ensemble, or target molecules, do not have to be added to the product for the purpose of detection but that molecules naturally present in the product can be referred to for the ensemble.

In a specific embodiment, the present invention relates to a method for providing an identifier for a product, the product comprising a product specific ensemble of molecules from a set of different distinguishable molecules, the method comprising the steps of: a) obtaining at least one sample of the product; b) analyzing the one or more samples in a manner using a set of molecules capable of recognizing and/or binding selected target molecules in generating a set of signals having strengths allowing determination of whether or not molecules from the set of different distinguishable molecules are to be considered to constitute part of the specific ensemble of molecules in the one or more samples, c) comparing signal strengths of the set to thresholds to determine a set of comparison results for molecules from the set of different distinguishable molecules, the comparison results in the set of results indicating whether and/or to what amount a respective of the different distinguishable molecule is to be considered present in the sample, molecule from the set of different distinguishable molecules being considered to constitute part of the specific ensemble of molecules in the sample or to be present in a specific amount depending on whether or not the respective signal strength exceeds one threshold or several thresholds; and d) compiling the set of comparison results into the identifier.

Accordingly, where there is a set of molecules that are different in a distinguishable manner and that are known to be found in a variety of products such that a specific product of the variety comprises a specific sub-set (or ensemble) of these molecules and/or comprises a specific sub-set of these molecules in specific concentrations or concentration ratios, it is possible to identify the product by specifying which molecules from the set of different distinguishable molecules can be found in the ensemble of molecules specific for a given product and which of these molecules cannot be found in the ensemble and/or by specifying to what extent the molecules of the ensemble can be found in the sample and/or what ratios of concentrations or signal strengths can determined.

In order to provide an identifier, a sample of the product is analyzed. This analysis is done by using a set of molecules capable of recognizing and/or binding selected target molecules and/or parts thereof. The target molecules that the set of molecules used in analyzing the sample is capable of recognizing and/or binding are molecules either constituting part of the set of different distinguishable molecules or are derived from these during storage or the analysis of the sample.

In another embodiment, the invention relates to a method for evaluating the authenticity of a food product, the method comprising the steps of (a) obtaining a sample of the food product; (b) generating a plurality of signals based on the presence and/or the amount of two or more target molecules in the sample obtained in step (a), wherein the generation of the plurality of signals comprises a sequencing method and/or a microarray assay, and comparing the strengths of a plurality of signals generated by one or more additional analytical methods to one or more thresholds; (c) compiling an identifier having a plurality of elements based on the plurality of signals generated in step (b); (d) determining one or more properties the identifier of the food product is expected to have to be authentic; (e) comparing the one or more properties determined in step (d) for the food product to the respective one or more properties of an identifier for a product that is known to be authentic; and (f) evaluating the authenticity of the candidate product based on the comparison made in step (e) wherein the method further comprises adjusting at least one threshold to the storage conditions of the candidate product prior to determining the identifier.

It is anticipated that when using a set of molecules capable of recognizing and/or binding selected target molecules that in turn can be found in at least some of the products or can be derived from at least some of the these products during analysis, some of the target molecule will be easier to detect in the products than others. For example, a target molecule may be part of the product-specific ensemble of molecules but it may be unstable over time so that it slowly decays. Thus, where the product is rather old, few target molecules will be found in a sample of a given size or volume and a signal generated during analysis might be rather weak. Accordingly, to allow determination of whether or not a given molecule from the set of different distinguishable molecules is found in the product, the signal strength may be compared to a threshold so that the comparison result is used rather than the absolute signal strength. This reduces determination errors due to influences that adversely affect the signal strengths by taking into account that processes might occur that might reduce signal strengths such as chemical oxidation of molecules, inhibition of reactions in some products, decay to due to adverse storage temperatures and so forth. Then, rather than looking at the absolute signal strengths, an identifier can even be compiled that comprises the results of the comparison, for example in form of a binary vector or binary matrix. However, other methods of providing an identifier are possible. In particular where establishing a library or database of known products, it might be advisable to store the signal strengths themselves rather than how they compare against a certain threshold. In this manner, if later on other reference products need to be added to the database that have respective signal strengths that while similar are still slightly different, it is easier to re-adapt the thresholds a candidate product must have so that it is considered to correspond to either the first or second reference product. Note that the signal strength stored might be a normalized signal strength, e.g. ranging between 0 and 100. Also, a ratio of signal strengths could be stored as identifier elements. Even where—rather than establishing a library—a candidate product is to be determined, it is possible to use values other than binary values, e.g. multibit levels indicative for a signal strength, such as 4 bit, 8 bit, 12 bit. representative of a signal strength and/or of a signal strength ratio.

It should be noted that molecules from the set of molecules capable of recognizing and/or binding selected target molecules will in some text passages be referred to as “binding molecules” for the sake of simplicity without excluding molecules capable of recognizing selected target molecules without binding.

In the preferred embodiment, the target molecules which the set of molecules is capable of recognizing and/or binding are molecules from the set of different distinguishable molecules and/or are derived from such molecules prior to or during analysis of the sample. The product for which the identifier is to be determined can be foodstuff, in particular a processed product. In a typical situation, the identifier is provided so that the authenticity of a product or other property of a product can be checked. In order to emphasize that frequently it is not known whether or not a product for which the identifier is provided is genuine or has the properties assumed and so forth, the product from which the sample has been obtained is frequently referred to as being a candidate product. In other words: Some products are examined to determine whether they are fake or genuine. These products may be termed “candidate products” hereinafter.

It will be understood that in a typical embodiment, the target molecules will be nucleic acid molecules, peptides or small or large molecules. These nucleic acid molecules, peptides or small or large molecules can be comprised in members of the microbiome and/or macrobiome of the sample and/or can be derived therefrom during storage and/or analysis. It will be understood that in particular, the nucleic acid molecules, peptides or small or large molecules that constitute target molecules that the set of molecules is capable of recognizing and/or binding, may be comprised in members of the microbiome of wine, in particular in the microbiome comprising fungi, yeast, bacteria and/or phages and/or may be derived from the members of the microbiome of the wine during storage and/or analysis and/or may be comprised in members of the macrobiome, in particular comprising plants, in particular vine and are derived from members of the macrobiome of the wine during storage and/or analysis.

It has to be noted that the terms “macrobiome” and “microbiome”, as used herein, also includes the remains of dead micro- or macroorganisms in a sample. For example, the macrobiome of a wine, for example, comprises all molecules in the wine that are derived from larger organisms such as plants. The microbiome of a wine, on the other hand, comprises all molecules that are derived from microorganism. In particular, a target molecule is said to be comprised in the microbiome of a wine, if the target molecule is a molecule that is part or that is derived from a microorganism. A target molecule that is part or is derived from a microorganism may end up in the wine by any means. In some instances, a target molecule that is part or that is derived from a microorganism may end up in the wine during the process of wine making, for example if the microorganism is in contact with the grapes or other parts of a plant. A target molecule is said to be comprised in the macrobiome of a wine, if the target molecule is a molecule that is part or that is derived from a larger organism, such as a plant.

The molecules capable of recognizing and/or binding selected target molecules may comprise one or multiple sets of molecules that are specific for genera, preferably species comprised in the macro- and/or microbiome in the sample of the product. The molecules capable of recognizing and/or binding selected target molecules may be or may comprise nucleic acid molecules, antibodies, or antibody-like polypeptides or peptides.

It should be noted that usually, the set of molecules capable of recognizing and/or binding selected target molecules will be brought into contact with the sample and/or a product obtained from the sample, for example after filtering, buffering, centrifugation, digestion and so forth.

In a preferred embodiment, the set of molecules capable of recognizing and/or binding selected target molecules comprises at least one nucleic acid molecule and the step of analyzing the sample comprises the use of hybridization of nucleic acid molecules to complementary sequences for DNA-microarray assays and/or for nucleic acid amplification methods and/or sequencing, in particular next generation sequencing.

It should be mentioned that a variety of DNA amplification methods may be employed such as multiplex PCR, real-time PCR, multiplex real-time PCR, Loop-mediated isothermal AMPlification (LAMP), Recombinase Polymerase Amplification (RPA) and rolling circle amplification. In a preferred embodiment, a PCR amplification multiplex method may be employed, e.g. multiplex real-time PCR. At least some nucleic acid molecules capable of binding and/or recognizing target molecules may target in a preferred embodiment the bacterial 16S rRNA genes. In a further preferred embodiment, immunoassay methods may be used in analyzing the sample or parts thereof, for example, but not exclusively, ELISA methods. Thus, in such an embodiment, it is preferred that the molecules capable of recognizing and/or binding selected target molecules comprise at least one antibody or antibody-like polypeptide, and the step of analyzing comprises the use of immunoassay methods, in particular a sandwich immunoassay method that makes use of a secondary antibody or antibody-like polypeptide for detection.

In certain embodiments, target nucleic acid molecules may be identified by sequencing. Any known sequencing method known in the art may be used for the identification of nucleic acid molecules in a sample. For example, target nucleic acids in a sample may be identified by Sanger sequencing with a sequence specific primer. Accordingly, multiple sequence specific primers may be used to identify the presence of two or more target nucleic acid molecules in a sample.

However, it is preferred in the present invention that target molecules in the sample are identified by “next-generation sequencing” or “high-throughput sequencing”. The terms “next-generation sequencing” or “high-throughput sequencing”, as used herein, refer to the so-called parallelized sequencing-by-synthesis or sequencing-by-ligation platforms currently employed by Illumine, Life Technologies, and Roche, etc. Next-generation sequencing methods may also include nanopore sequencing methods such as that commercialized by Oxford Nanopore Technologies, electronic-detection based methods such as Ion Torrent technology commercialized by Life Technologies, or single-molecule fluorescence-based methods such as that commercialized by Pacific Biosciences.

In certain embodiments, it is attempted to assign the target nucleic acids, or nucleic acid molecules that have been obtained from target nucleic acids during analysis, for example by amplification, that are identified by sequencing to a specific genus or species. For example, primers may be designed that only hybridize with a nucleic acid molecule from a specific species or a specific genus.

Next generation sequencing allows the sequencing of a substantial fraction of nucleic acids in a sample or even multiple samples in a single sequencing run. For this, target nucleic acid molecules, or nucleic acids that have been obtained from target nucleic acid molecules during analysis, for example by amplification, may be attached to an adapter and sequenced using universal sequencing primers. This approach has the advantage that no detailed sequence information about the nucleic acid molecules that are expected to be comprised in the sample(s) are required beforehand and that the obtained sequences can then be assigned to a specific species or genus later on, preferably supported by bioinformatic approaches.

When attempting to assign reads that correspond to target nucleic acid molecules in a sample to a species or genus, any read that is obtained by next-generation sequencing may be assigned to a specific species or genus, for example by mapping on a reference genome. Alternatively, two or more overlapping reads may be assembled and then mapped on a reference genome. Sequencing may be performed with the entire nucleic acid content of a sample. Further, the nucleic acid content of a sample may be pre-amplified in an unspecific manner before sequencing. The pre-amplified nucleic acids may also be subjected to sequence-specific amplification step. For example, it may be preferable to amplify specific genes or parts of the genome that are known to be present in a large group of species or substantially an entire domain and that can be used to identify the species or genera of the organisms the nucleic acids in the sample have been derived from. In certain embodiments, sequencing the 16S rRNA genes may be performed to identify the species or genera of the organisms the nucleic acids in the sample have been derived from.

The 16S rRNA gene is highly conserved between different species of bacteria and archaea. It is suggested that the 16S rRNA gene can be used as a reliable molecular clock because 16S rRNA sequences from distantly related bacterial lineages are shown to have similar functionalities. In addition to highly conserved primer binding sites, 16S rRNA gene sequences contain hypervariable regions that can provide species-specific signature sequences useful for identification of bacteria. As a result, 16S rRNA gene sequencing has become prevalent in medical microbiology as a rapid and cheap alternative to phenotypic methods of bacterial identification. Although it was originally used to identify bacteria, 16S sequencing was subsequently found to be capable of reclassifying bacteria into completely new species, or even genera. It has also been used to describe new species that have never been successfully cultured. With next-generation sequencing simultaneous identification of thousands of 16S rRNA sequences is possible within hours, allowing metagenomic studies. The bacterial 16S gene contains nine hypervariable regions (V1-V9), ranging from about 30 to 100 base pairs long, that are involved in the secondary structure of the small ribosomal subunit. The degree of conservation varies widely between hypervariable regions, with more conserved regions correlating to higher-level taxonomy and less conserved regions to lower levels, such as genus and species.

Accordingly, in certain embodiments, nucleic acids that have been derived from specific bacterial species and/or genera may be identified by sequencing 16S rRNA genes in a sample. In certain embodiments, the nucleic acids in the sample may be pre-amplified in a non-specific manner to increase the nucleic acid content in the sample. In certain embodiments, the nucleic acids in the sample may be pre-amplified using reagents from the REPLI-g Single Cell Kit from Qiagen. In certain embodiments, the (pre-amplified) 16S rRNA genes in the sample or parts thereof may be specifically amplified by PCR before the sequencing step. In certain embodiments, the V3 and/or V4 hypervariable regions may be specifically amplified before the sequencing step. In certain embodiments, the primers V3 (SEQ ID NO.11) and V4 (SEQ ID NO:12) may be used for the amplification of the V3-V4 hypervariable region. In certain embodiments, pre-amplified and/or amplified nucleic acid molecules may be attached to one or more adapters. In certain embodiments, pre-amplified nucleic acid molecules may be fragmented before attaching the one or more adapters. In certain embodiments, the adapters comprise one or more barcodes, for example the Illumina i5 and/or i7 barcodes. In certain embodiments, a unique i5/i7 combination is used for each sample that is sequenced in the same run. In certain embodiments, the adapters are attached to the nucleic acid molecules by PCR. In certain embodiments, the nucleic acid molecules attached to the one or more adapters are denatured before sequencing.

Attaching a unique barcode combination to the nucleic acids that have been obtained from the same sample allows parallel sequencing of multiple samples in the same run. In the next-generation sequencing run, a plurality of sequences is obtained. These sequences may be compared to a library of reference genomes to identify the organism the nucleic acid is derived from. Mapping the sequences on reference genomes may be done manually or may be done automatically with a suitable software. In certain embodiments, the SequenceHub platform (Illumina) is used to automatically assign individual reads to specific species or genera. In certain embodiments, the 16S metagomic workflow may be used for assigning sequences that correspond to 16S rRNA genes to specific species or genera. In certain embodiments, the kraken workflow may be used for assigning sequences from whole genome sequencing approach to specific species or genera.

Compiling the comparison results or signal strengths into an identifier can be effected by a variety of measures.

Within the present invention, an identifier comprises information about the presence and/or the concentration of a plurality of target molecules in a sample. In particular, each element of the identifier comprises information whether and/or to which extent a specific target molecule is present in a sample. In certain embodiments, a target molecule is said to be present in a sample if a signal can be generated that corresponds to this target molecule. In particular, a target molecule is said to be present in a sample, if the strength of a signal is above a specific threshold. A threshold that is used to determine if a target molecule is present in a sample may be defined by different means.

In general, a threshold may be a pre-defined threshold or may be a threshold that is determined based on the strength of one or more signals. In addition, the same threshold may be used for each target molecule that is analyzed or a specific threshold may be determined for each target molecule that is analyzed. Also, a combination of these approaches is envisioned in the present invention. That is, the signals for one set of target molecules may be compared to a pre-defined threshold and the signals for another set of target molecules may be compared to one or more specific thresholds that have been determined based on generated signals.

In a certain embodiment, generating the signals comprises a sequencing step or a microarray assay. For example, the plurality of signals may be generated by next-generation sequencing. Next-generation sequencing results in the generation of a plurality of reads that can be subsequently mapped on one or more reference genomes. A “target molecule”, as used in the present invention, may be a particular gene or an entire genome of a specific species or a genus comprising multiple species. When referring to nucleic acid target molecules, the term “target molecule” comprises both nucleic molecules that are originally present in a sample and nucleic acid molecules that have been obtained from these nucleic acid molecules by amplification. In certain embodiments, the gene may be a gene encoding a bacterial 16S rRNA. Accordingly, each read from a next generation sequencing run that can be mapped to a particular gene or genome of the same species or genus will contribute to the same signal, even if the reads map to different parts of the gene or genome and are thus different in sequence. Accordingly, a “signal” may correspond to the number of reads that have been mapped to a gene or genome of a specific species or genus, i.e. a target molecule.

If a target molecule is present and/or to which extent a target molecule is present in a sample may be determined based on the strength of a signal that corresponds to said target molecule. For example, the strength of the signal may correspond to the number of reads from a next-generation sequencing run that can be assigned to a specific target molecule.

In certain embodiments, a target molecule is defined to be present in a sample, if at least a certain number of reads are detected that correspond to this target molecule. Thus, a target molecule may be determined to be present in a sample, if at least 1, at least 10, at least 25, at least 50, or at least 100 reads are detected that correspond to said target molecule. Accordingly, the determination if a specific target molecule is present in a sample may be based on the absolute number of reads from a next-generation sequencing run.

Alternatively, one or more thresholds may be defined for a target molecule based on a signal strength or a plurality of signal strengths that correspond to a target molecule in a sample from a product that is known to be authentic. For example, a threshold may be defined that is lower than the signal strength or the mean signal strength that has been determined for a target molecule in a product that is known to be authentic. In this case, a target molecule is determined to be present in a sample if the signal strength for the same target molecule is higher than the defined threshold for this target molecule. Alternatively, a higher and a lower threshold may be defined for a target molecule based on the signal strength(s) that has/have been determined for a target molecule in one or more samples from a product that is known to be authentic. In certain embodiments, the upper and lower thresholds may be defined based on the upper and lower boundaries of a confidence interval that has been determined for a plurality of signals corresponding to the same target molecule, preferably wherein the signals have been generated from samples that have been obtained from the same food product. In this case, a target molecule is determined to be present in comparable amounts in a sample of a candidate product and a sample of a product that is known to be authentic, if the signal strength that corresponds to this target molecule is above the lower threshold and below the upper threshold.

Alternatively, the determination if a target molecule is present in a sample and/or to which extent it is present in a sample may be based on the relative abundance of a target molecule.

For example, the strength of a signal may be normalized to an internal or external reference. Due to the variation in the composition of a food product, such as varying pH or alcohol content, or due other small variations in the experimental setup, variations in signal strength may be observed. In case of next-generation sequencing, this means that varying signal strengths for the same target molecule may be obtained for samples comprising the same or similar amounts of said target molecule. In this case, the number of reads may be normalized to an external or internal standard. An “internal standard”, as used herein, is a molecule that is naturally present in substantially all samples in a same or similar amount. Preferably, an external standard is used for the normalization of signals, as the concentration of an external standard can be adjusted more reliably than the concentration of an internal standard. Preferably, the internal or external standard is a nucleic acid molecule. For example, an external standard may be a nucleic acid molecule with a known sequence that is added to the sample prior to analyzing the sample. Within the present invention, an external standard may be added before or after a pre-amplification and/or amplification step. For normalizing a signal, the signal that has been obtained for a specific target molecule may be divided by the signal that has been obtained for the internal or external standard.

In a preferred embodiment of the invention, a single sample of the product is obtained that is sufficient to determine for each single molecule from the set of different distinguishable molecules whether or not such single molecule is to be considered to constitute part of the specific ensemble of molecules in the sample. In other words, one single sample is obtained and a complete analysis thereof is done.

However, it would also be possible to obtain a first sample of (smaller) volume, determine whether or not a first molecule from the set of different distinguishable molecules can be found in the first small volume sample, and to then compare the respective signal strength to a given threshold. This result could be compiled into a (coarse or partial) identifier. Then, depending on how the partial identifier—having e.g. a limited number of elements—compares to the respective identifier elements of one or more products known to be genuine, a further sample could be obtained for further tests. Also, some but not all molecules from the set of different distinguishable molecules could be analyzed using one small volume sample, the resulting signal strengths being e.g. compared against thresholds and the comparison results compiled into an identifier that while not allowing a complete identification in the most precise manner possible still allows to determine some properties of the product. It is noted that on some occasions, a determination of an identifier might suffice, even without referring to a library. For example, where a user has several bottles allegedly containing the same wine, it can be determined whether they all have the same or at least similar descriptor behavior.

A single comparison result or partial identifier can be used in a first iteration step checking whether or not a given candidate product is to be considered authentic in view of the partial result. Thus, compiling the set of comparison results would be done in an iterative manner so that the identifier will be altered, typically extended, with each iteration.

Note that even where the analysis is effected in a non-iterative manner, that is by looking at all signals allowing determination of whether or not and/or to what extent each single molecule from the set of different distinguishable molecules is to be considered part of the specific ensemble molecules in the product prior to compiling and/or using the identifier, it would be possible to obtain a plurality of (as need be: smaller) samples from any given product.

For example, where a batch of wine bottles allegedly having the same origin and being from the same vintage is to be analyzed, a plurality of samples would be obtained by obtaining one sample from every bottle. Also, where the product is a food product that is non-homogeneous, obtaining a plurality of samples might be useful. In general, the identifier may be a matrix, for example a binary matrix of size (m×1) having m bits with m corresponding to the number of distinguishable different molecules in the set of different distinguishable molecules. It will be understood that where a plurality of n samples is analyzed, for example n samples, the binary matrix may be an (m×n) matrix.

Also, it is noted that while the matrix may be a binary matrix having a number of bits such as m bits for an (m×1) matrix, it is easily possible to transform the binary matrix or the data contained therein into some other (non-matrix) data, for example by using encryption, a data compression, preferably an information loss-free data compression, and so forth.

It will be obvious that preferably the set of thresholds to which the set of signal strengths is compared is determined with a view on a set of signal strengths obtained for a comparable product known to be genuine. For example, where it is to be checked whether a batch of wine bottles has the precise vintage and origin that is stated on the label of the wine, and where the exact same wine is available as a product known to be genuine, it is possible to first determine the signal strengths obtained during analysis of the genuine product and to then compare the thresholds thus obtained for each signal to the signal strengths obtained during analysis of samples from the candidate bottles. When doing so, it is possible to account for variations of the signal strengths, for example due to a certain degree of noise or due to storage conditions.

It should be noted that some of the molecules of the set of different distinguishable molecules may be unstable to some extent. Basically, while every different distinguishable molecule from the set of molecules can be expected to be subjectable to conditions where it decays, the different molecules might be affected more or less by simply storing the product for a prolonged period even though the conditions of storage may correspond to conditions recommended. In other words, for some molecules a degradation or decay will be stronger than for other molecules and this could be taken into account. It might thus be helpful to specify (not just) a given signal strength but also how the signal strength is expected to be effected by future aging. Accordingly, degradation kinetics and its effect on target molecule availability and/or on the target molecule amount in a sample can be considered. This can be done by considering ratios of concentrations of different target molecules.

For example, certain wines of very high quality can be stored for long periods such as for several decades or centuries. This may lead to differences of the signal strength for a given signal (or correspondingly a given molecule from the set of different distinguishable molecules) over several years. If the authenticity of a candidate product is to be checked by comparing the identifier of a candidate product to the identifier of a product known to be genuine, it is thus useful to adapt the thresholds to the long storage period.

It should also be noted that reasons other than prolonged storage periods may exist that make adaption of the threshold useful. For example, a case may exist where a candidate product cannot be compared against an identical product known to be genuine. This might for example be the case where an identifier for a rare, expensive, very old wine of a given vintage cannot be found in a library but where wines from the same origin (for example the same producer) and also being very old, but from different vintages are found in a library. Here, it should be expected that molecules in the set of different distinguishable molecules relating to the vine can be found in the candidate product as well so that the signature relating to the vine is identical, while only partial identity with respect to different distinguishable molecules of the set stemming from members of the microbiome, for example fungus, yeast and so forth is given. Thus, at least an estimate or a probability can be given that the candidate product is authentic.

However, with respect to the thresholds of the respective signals relating to molecules that constitute part of the ensemble of the candidate product, it should be noted that wines from different vintages may have a different content of alcohol, a different acidity and so forth, so that the overall ensemble of molecules will also differ from vintage to vintage. This may affect the stability of the molecules in the specific ensemble and it may also influence the extent of inhibition of the detection of molecules in the step of analyzing. Here, again, using a threshold rather than referring to the absolute strength of signals allows taking such effects into account in a useful manner even though these effects cannot be fully determined. It should be understood by a person skilled in the art that using a set of thresholds allows to check the authenticity of a candidate product in particular in cases where the data base is rather sparse, that is, where the library of products known to be genuine does not comprise every possible candidate product and/or where the conditions a candidate product has been subjected to over time are not fully known and/or the influence of such conditions is not fully understood.

From the above, it is obvious that the invention also relates to a method of evaluating the authenticity of a candidate product comprising the steps of providing an identifier for the candidate product according to one of previous embodiments, determining from a library of information relating to products known to be genuine one or more properties the identifier of an authentic candidate product is expected to have, comparing the one or more properties determined from the library to the respective one or more property of the identifier of the candidate product, judging that the candidate product should not be considered authentic if one or more properties the identifier of the candidate product has does not compare favorably to the one or more properties the identifier should have according to information relating to a genuine product.

The judgment as to whether or not a candidate product is to be considered genuine can be made in different ways depending on the information in the library and/or the identifier therein. For example, where the identification is obtained by a comparison of signal strengths against thresholds, resulting in a binary identifier or an identifier having differentiated ranges, such as high/middle/low, a full 1:1 correspondence of each element in the identifier of a candidate product to the identifier of a reference product might be required so that the candidate product is considered genuine. However, it is also possible to compile the signal strengths into a fine granular vector (such as a vector having one 8 bit-component for each signal strength considered). Then, a (scalar) product between the vector identifying the candidate product and the corresponding reference product vector can be considered without requesting a 1:1 correspondence. Note that if a scalar product is determined, the different vector components corresponding to the different identifier elements might be weighted differently. The candidate product can be identified in view of such a product, e.g. by considering for which reference product identifier the largest result by multiplication with the candidate product identifier is obtained.

It should be noted that in certain cases additional tests might be indicated to evaluate the authenticity of a candidate product. For example, it has been known in the past to illegally adulterate cheap wines using toxic di-ethylene glycol to make the wine appear sweeter and more full-bodied. If in such cases, a check would be made relying only on nucleic acids as the molecules in the set of different distinguishable molecules, the identifier would correspond to the identifier expected according to the labeling. However, the candidate product would still be far from authentic as a wine poisoned by adulterating should not be considered a product at all. Thus, in very rare circumstances, the method of the present invention might be combined with additional evaluation methods known per se in the art, e.g. analyzing for the general chemical content as known per se in the art. This holds for very expensive wines as well, in particular in case where a danger of adulterating exists.

It has been indicated above that for high quality wines of very old vintage, it may be next to impossible to build a library of products known to be genuine for each possible candidate product. Therefore, it is advantageous to have an embodiment that allows evaluating of the authenticity of a candidate product even based on a sparse database.

In this respect, it is advantageous to make use of the observation that while even wines from the same producer differ from vintage to vintage, the differences actually observed between wines of different vintages but from the same producer or between wines from different producers but from the same geographical region and/or from the same grape reflect certain identical properties. For example, in a wine frequently members of both the macrobiome, for example of the grapes, and members of the microbiome, such as fungi and yeasts, can be found. It has been found that for a given geographical region and/or a given producer, the same wines, for example a Burgundian, Dornfelder, Pinot Noir and so forth will have closely corresponding ensembles, in particular macrobiome ensembles, although the microbiome will be different from year to year resulting in differences with respect to the corresponding set of different distinguishable molecules, larger differences existing for different producers/regions. Here, it has been found that while vintages of a given wine from one producer have some variation in the microbiome from vintage to vintage, this variation typically is smaller than the differences to the microbiome of a different producer of the same wine sort in the same geographical region. It is at present not clear why this is the case. However, a possible explanation might be that some pathogens observed might be present dependent on the weather during growth or harvest of the grapes and/or might have accidentally contaminated the product. Nonetheless, this surprising result allows to at least estimate whether the probability of a candidate product being authentic or not is high or whether this probability is low even though neither the exact conditions of storage are known nor an identical genuine product is available.

Again, judgment whether or not a candidate product is genuine can be made in several ways, depending inter alia on the way the identifier is compiled. It will be understood that an embodiment is preferred where in case of a sparse database, a probability is determined and different weights are assigned to different parts of an identifier.

Thus, an embodiment is preferred wherein if for a candidate wine no identical wine from the same producer and the same vintage is included in the library, the one or more property the identifier of the candidate wine is expected to have is evaluated with a view on an importance of the one or more property, in particular such that to one or more properties relating to members of the macrobiome of the wine, in particular comprising plants, in particularly vine, an importance higher than the importance of one or more properties relating to members of the microbiome of the wine, in particular in the microbiome comprising fungi, yeasts, bacteria and/or phages is assigned and wherein preferably, in judging the candidate product to be authentic, a weight is assigned to the properties (or the identifier elements reflecting these properties) dependent on their importance.

It has been indicated above that the assumption of authenticity of a candidate product can be rejected in an iterative manner. This allows elimination of candidate products as authentic in a particularly inexpensive way. Thus, an embodiment is preferred wherein rejecting the assumption of a candidate product being authentic is attempted in an iterative manner, comprising the steps of providing in a first iterative step a first part of the identifier information of the candidate product, attempting to falsify that the candidate product is authentic based on one or more properties of the first part of identifier information, providing a further part of the identifier information of the candidate product in case the candidate product cannot be falsified in a previous step, attempting to falsify that the candidate product is authentic based on the further information, in particular repeating the iteration until either the assumption of authenticity is falsified or identifier information relating to all molecules of the set of different distinguishable molecules has been evaluated.

The identifier provided by the invention can be used in different ways. In a first example, it might be necessary for a collector of expensive old wines to store his collection outside of his home. Here, the collector might have a high interest to verify that the bottles stored by a 3rd party are not adulterated. One method would involve taking a sample of a bottle stored, providing the identifier and storing the identifier. Then, later on, when the owner of the expensive wine wants to check whether the bottle has been adulterated, a further sample is taken and the identifier is determined. This identifier should, of course, be identical to the identifier previously determined for the same bottle. Thus, the identifier can be used even without a library of information relating to a large number of different genuine products.

However, another application of the present invention is determination of whether or not an expensive wine newly acquired by a connoisseur or collector of wines is genuine or not. In this case, information is needed that allows to authenticate the candidate product. As such authentication should be cheap on the one hand and specific on the other hand, it usually is advisable to select from a very large variety of different distinguishable molecules, for example from a large variety of different nucleic acids found in a large number of different wines, a minimum set that is particularly specific or that relates to different distinguishable molecules that can best be analyzed, for example because the selected molecules are least affected by the differences in the wine fluid they are comprised in and/or because they are most stable. Note that even a minimum set need not be the set having the absolute smallest number of different molecules. The absolute smallest number to distinguish (2\hat{ }\hat{ }n) different reference products using binary identifiers would be n; accordingly, determining e.g. for 5 different molecules whether they are present in a product might suffice to distinguish 32 different products. However, overall analysis might become easier if more than 5 signal strengths are considered. Also, later on, when expanding the data base, this might help to avoid that additional signal strengths need to be determined.

In order to identify those molecules in the variety of different distinguishable molecules found in a large number of wines, the invention suggests to analyze samples from a large number of genuine products using a large plurality of molecules capable of recognizing and/or binding selected target molecules to determine a plurality of signal strengths relating to the presence, absence and/or concentrations of molecules from the plurality of distinguishable molecules. Those molecules that are particularly suitable to discriminate one genuine product from other genuine products can be determined; preferably, a minimum set of molecules is selected, that is a set of molecules that has a minimum number of molecules but that still allows discrimination of all considered genuine products from each other.

It will be understood that where the number of different genuine products is small, only a small set of molecules needs to be determined, that is very few molecules need to be selected from the plurality of different distinguishable molecules. However, as the database grows, occasions may occur where additional molecules may need to be added to the set of molecules so as to allow continued discrimination of a growing number of genuine products. Then, for these molecules, the thresholds of signal strengths can be established and will be included in the library of information so that when determining a signal strength relating to one or more specific molecule from the selected set, it can be determined whether or not such a given molecule from the set is considered to be present in a sample based on the signal strength by simple comparison to the threshold.

It will be understood that where the identifier is compiled in a manner comprising signal strengths and thresholds, it is not necessary to store identical threshold information in the library. Rather, it would for example be possible to store a threshold in the library obtained by a given process together with the date the signal has been measured, and/or the vintage of a wine and/or kinetic data relating to the stability of a molecule the threshold relates to. In this manner, if a sample from the same product, that is the same wine from the same vintage, is analyzed later on, a decay of the molecule in the ensemble can be taken into account and where a similar wine of different vintage is to be analyzed, the threshold can be corrected as well. It should be noted that additional properties of molecule stability can also or alternatively be included in the library, for example relating to the stability of the molecules in wine matrixes of different alcohol content and/or of different pH. Where such information is available, it can be retrieved together with the threshold when comparing signal strengths during provision of an identifier and an appropriate threshold can be determined. As an alternative, it is not necessary to include additional information such as kinetic data relating to the stability of a given molecule from the set in the very library. Rather, such information allowing adaption of thresholds could be included in a separate library and retrieved separately when an identifier for a candidate product is to be provided. Also, an in-silico determination would be possible from suitable data.

It should be noted that while generally the set of molecules distinguishable from each other but specific for a genuine product in an ensemble should be as small as possible. There might be cases where several distinguishable molecules each could be used as part of the set. In that case, it will be understood that the most stable molecule could be selected, a molecule resulting in the best signal-to-noise ratio of signal strengths generated during analysis could be selected and/or a molecule being least effected in a different way by different chemical conditions of the fluid it is contained in could be selected.

It will be understood that in a preferred embodiment, a kit can be provided for performing the method, for example comprising a system on a chip or the like and/or a container for a sample of the product and instructions how to execute the method and/or how to have the method executed, e.g. by sending it to a specific laboratory or contact address requesting a specific analysis, e.g. by including a corresponding voucher. Also, instead of the instructions themselves, a data carrier comprising the instructions and/or a link or other information where to download instructions from could be included in the kit.

It is also possible to provide a kit or a device comprising primers for the detection of components of the macrobiome and/or microbiome in a manner allowing determination of an identifier according to the method of the invention. Furthermore, a kit or a device comprising a fluidic array with one or more primer(s) to perform multiplexed PCR in a manner allowing determination of an identifier according to the method of the invention and/or comprising a microarray with one or more oligonucleotide(s) to perform hybridization assays in a manner allowing determination of an identifier according to the method of the invention is suggested.

### Definitions

The term “microbiome” relates to a community of commensal, symbiotic or pathogenic microorganisms, and their genomes, found in and on all multicellular organisms, i.e. plants and animals. A microbiome includes fungi, yeasts, bacteria, viruses, phages, archaea, protists, both, living and nonliving.

By contrast thereto, the term “macrobiome” relates to the macroflora and macrofauna and their genomes, i.e. to plants and animals including human.

A microorganism, or microbe, is a microscopic organism, which may exist in its single-celled form or in a colony of cells.

The term “nucleic acid sequence” refers to the sequence of nucleotides in a nucleic acid. Nucleic acids consist of a chain of linked units called nucleotides. Each nucleotide consists of three subunits: a phosphate group and a sugar (ribose in the case of RNA, 2′-deoxyribose in DNA) make up the backbone of the nucleic acid strand, and attached to the sugar is one of a set of nucleobases. In case of DNA, the nucleobases are basically adenine A, guanine G, thymine T and cytosine C, and in case of RNA, thymine is replaced by uracil U. By convention, the base sequence is noted from the 5′ end to the 3′ end of the strand, in the same direction in which the polymerase synthesizes the nucleic acid from nucleotides. The sequence has capacity to represent information. Biological deoxyribonucleic acid represents the information which directs the functions of a living being. The nucleobases are important in base pairing of strands to form higher-level secondary and tertiary structure such as the famed double helix. Thereby, the base pairs are A=T and G≡C. If one strand of the double-stranded DNA is considered the sense strand, then the other strand, considered the antisense strand, will have the complementary sequence to the sense strand.

In biological systems, nucleic acids contain information which is used by a living cell to construct specific proteins. The sequence of nucleobases on a nucleic acid strand is translated by cell machinery into a sequence of amino acids making up a protein. Each set of three bases, called a codon, in principle corresponds to a single amino acid, and there is a specific genetic code by which each possible set of three bases corresponds to a specific amino acid.

The central dogma of molecular biology outlines the mechanism by which proteins are constructed using information contained in nucleic acids. DNA is transcribed into mRNA molecules, which translocate to the ribosome where the mRNA is used as a template for the construction of the protein strand. Since nucleic acids can bind to molecules with complementary sequences, there is a distinction between “sense” sequences which code for proteins, and the complementary “antisense” sequence which is by itself nonfunctional, but can bind to the sense strand.

The term “nucleic acid amplification” relates to the artificial increase in the number of copies of a particular DNA fragment. Nucleic acid amplification methods can be used to overcome the limitations of direct probe hybridization assays. Nucleic acid amplification is a pivotal process in biotechnology and molecular biology and has been widely used in research, medicine, agriculture and forensics. Polymerase chain reaction (PCR) was the first nucleic acid amplification method developed and until now has been the method of choice since its invention by Mullis (Mullis K B Sci Am. 1990 April; 262(4):56-61, 64-5). PCR is the preferred method for application oriented fields involving nucleic acid amplification for its simplicity, easier methodology, extensively validated standard operating procedure and availability of reagents and equipment. However, PCR has a good number of limitations, including high cost of equipment, contamination chances, sensitivity to certain classes of contaminants and inhibitors, requirement of thermal cycling etc. (Fakruddin M. Loop mediated isothermal amplification—An alternative to polymerase chain reaction (PCR) Bang Res Pub J. 2011; 5:425-39). These limitations gave birth to alternative methods such as loop mediated isothermal amplification (LAMP) Notomi T, Okayama H, Masubuchi H, Yonekawa T, Watanabe K, Amino N, Hase T, Nucleic Acids Res. 2000 Jun. 15; 28(12):E63), nucleic acid sequence based amplification (NASBA) (Compton J, Nature. 1991 Mar. 7; 350(6313):91-2), self-sustained sequence replication (3SR) (Guatelli J C, Whitfield K M, Kwoh D Y, Barringer K J, Richman D D, Gingeras T R, Proc Natl Acad Sci USA. 1990 October; 87(19):7797), rolling circle amplification (RCA) etc., most of which are isothermal nucleic acid amplification methods obviating the need of thermal cycler (see review of Md Fakruddin, Khanjada Shahnewaj Bin Mannan, Abhijit Chowdhury, Reaz Mohammad Mazumdar, Md. Nur Hossain, Sumaiya Islam, and Md. Alimuddin Chowdhury, J Pharm Bioallied Sci. 2013 October-December; 5(4): 245-252). Further amplification methods are strand displacement amplification (SDA) and ligase chain reaction (LCR).

The term “DNA sequencing” and/or “RNA sequencing” relate to the process of determining the precise order of nucleotides within a DNA molecule and a RNA molecule, respectively. Many DNA and RNA sequencing methods are known to the skilled person. Maxam-Gilbert sequencing (Maxam A M, Gilbert W (February 1977), “A new method for sequencing DNA”, Proc. Natl. Acad. Sci. U.S.A. 74 (2): 560-4)) was the first widely adopted method for DNA sequencing, and, along with the Sanger dideoxy method (Sanger F; Coulson A R (May 1975), “A rapid method for determining sequences in DNA by primed synthesis with DNA polymerase”, J. Mol. Biol. 94 (3): 441-8; Sanger F; Nicklen S; Coulson A R (December 1977), “DNA sequencing with chain-terminating inhibitors”, Proc. Natl. Acad. Sci. U.S.A. 74 (12): 5463-7), represents the first generation of DNA sequencing methods. Maxam-Gilbert sequencing is no longer in widespread use, having been supplanted by next-generation sequencing methods.

The term “next generation sequencing” refers to high-throughput sequencing methods which apply to genome sequencing, genome resequencing, transcriptome profiling (RNA-Seq), DNA-protein interactions (ChIP-sequencing), and epigenome characterization (de Magalhães J P, Finch C E, Janssens G (2010). “Next-generation sequencing in aging research: emerging applications, problems, pitfalls and possible solutions”. Ageing Research Reviews. 9 (3): 315-23). The high demand for low-cost sequencing has driven the development of high-throughput sequencing technologies that parallelize the sequencing process, producing thousands or millions of sequences concurrently (Grada A (August 2013), “Next-generation sequencing: methodology and application”, J Invest Dermatol. 133 (8): 1-4; Hall N (May 2007), “Advanced sequencing technologies and their wider impact in microbiology”, J. Exp. Biol. 210 (Pt 9): 1518-25; Church G M (January 2006), “Genomes for all”. Sci. Am. 294 (1): 46-54). High-throughput sequencing technologies are intended to lower the cost of DNA sequencing beyond what is possible with standard dye-terminator methods (Schuster S C (January 2008), “Next-generation sequencing transforms today's biology”, Nat. Methods. 5 (1): 16-18). In ultra-high-throughput sequencing as many as 500,000 sequencing-by-synthesis operations may be run in parallel (Kalb, Gilbert; Moxley, Robert (1992), Massively Parallel, Optical, and Neural Computing in the United States. IOS Press. ISBN 978-90-5199-097-3; ten Bosch J R, Grody W W (2008). “Keeping Up with the Next Generation”, The Journal of Molecular Diagnostics, 10 (6): 484-92; Tucker T, Marra M, Friedman J M (2009), “Massively Parallel Sequencing: The Next Big Thing in Genetic Medicine”, The American Journal of Human Genetics. 85 (2): 142-54). Further high-throughput sequencing methods are described and compared in Quail M A, Smith M, Coupland P, Otto T D, Harris S R, Connor T R, Bertoni A, Swerdlow H P, Gu Y (1 Jan. 2012). “A tale of three next generation sequencing platforms: comparison of Ion Torrent, Pacific Biosciences and illumine MiSeq sequencers”. BMC Genomics. 13 (1): 341 and Liu L, Li Y, Li S, Hu N, He Y, Pong R, Lin D, Lu L, Law M (1 Jan. 2012). “Comparison of Next-Generation Sequencing Systems”. Journal of Biomedicine and Biotechnology. 2012: 1-11. Any sequencing method as known from the state of the art can be used for the present invention and the above list is not limiting.

The term “real-time multiplex PCR” refers to the use of polymerase chain reaction to amplify several different DNA sequences simultaneously (as if performing many separate PCR reactions all together in one reaction). This process amplifies DNA in samples using multiple primers and a temperature-mediated DNA polymerase in a thermal cycler. The primer design for all primer pairs has to be optimized so that all primer pairs can work at the same annealing temperature during PCR. Multiplex-PCR consists of multiple primer sets within a single PCR mixture to produce amplicons of varying sizes that are specific to different DNA sequences. By targeting multiple sequences at once, additional information may be gained from a single test run that otherwise would require several times the reagents and more time to perform. The different amplicons may be differentiated and visualized using primers that have been dyed with different colour fluorescent dyes. Results are obtained in real-time (see for instance Richard Molenkamp, Alwin van der Ham, Janke Schinkel, and Marcel Beld, Biochemica No. 3, 2007, p. 15-17).

The term “nucleic acid microarray” (also commonly known as DNA chip or biochip) refers to a collection of very small oligonucleotide spots attached to a solid surface. DNA microarrays can be used to measure the expression levels of large numbers of genes simultaneously or to genotype multiple regions of a genome. The core principle behind microarrays is hybridization between two DNA strands, or a DNA and a RNA strand, the property of complementary nucleic acid sequences to specifically pair with each other by forming hydrogen bonds between complementary nucleotide base pairs. A high number of complementary base pairs in a nucleotide sequence means tighter non-covalent bonding between the two strands. After washing off non-specific bonding sequences, only strongly paired strands will remain hybridized. Probe-target hybridization is usually detected and quantified by detection of fluorophore-, silver-, or chemiluminescence-labeled targets to determine relative abundance of nucleic acid sequences in the target. Nucleic Acid microarrays often use relative quantification in which the signal intensity of a spot is compared to the signal intensity of the same spot under a different condition or to a different spot on the same chip, and the identity of the spot is known by its position.

The term “threshold value” refers to a value which determines the presence or absence of specific target molecules. For example, a high concentration of target molecules will usually result in a strong signal such as a strong band in a PCR process whereas a low concentration will result in a weak band in a PCR process. However, it will be understood that a useful threshold value must take into account that for some molecules even though their concentration in the product is high, detection is particularly difficult and hence the signal may be rather weak. Also, it will be understood that different methods of generating a signal may result in signals other than strengths of bands; for example, a photogrammetric analysis of a band pattern might result in a gray value corresponding to a digital value. Also, using an appropriate threshold value it can be checked whether an amount of the target molecule has some upper or lower boundary.

It should be noted that according to the present invention, the product is identified in view of molecules considered to constitute part of an ensemble of molecules found in the product. This is done using (other) molecules binding to selected target molecules. The target molecules may be those molecules that are found in the product and in the ensemble. However, it is also possible to derive the target molecules from the molecules in the ensemble, e.g. by digestion, amplification of DNA sequences and the like. The target molecules might be derived from the molecules in the ensemble basically at any stage prior to signal detection, e.g. during storage due to oxidation or during analysis.

Within the present invention, it is preferred that the signals that correspond to specific target molecules are generated by next-generation sequencing and/or microarray assays. However, the method of the invention may further be complemented by addition analytical methods to refine the authentication results. For example, additional target molecules such as further nucleic acids, peptides, carbohydrates, and/or small or large molecules may be analyzed for the authentication of a food product. For example, other nucleic acids may be analyzed and/or compared by PCR-based methods. In addition, nucleic acids, peptides, carbohydrates and/or small or large molecules may be analyzed by mass spectrometry, nuclear magnetic resonance (NMR) and/or immunoassays such as ELISA. The results from these complementary methods may be further compiled into the identifier to generate further elements and refine the authentication process.

The stability of molecules in a food product can vary significantly between different molecules. For example, nucleic acids are known to be rather stable molecules and their decay behavior is largely independent of the sequence of a nucleic acid. Other molecules in a food product, however, may be highly instable and have a much shorter half-life. Accordingly, a ratio may be determined between a signal that has been generated for a rather stable target molecule, such as a nucleic acid, and a signal that has been generated for a rather unstable target molecule with one or more additional analytical methods. This ratio may then be compared to ratios from other samples or to one or more thresholds. By comparing ratios between rather stable and rather unstable target molecules in samples from food products, it may be possible to further refine the authentication and/or identification of a food product. For example, by determining the ratio of a rather stable target molecule, such as a nucleic acid molecule, and a rather unstable molecule, it may be possible to determine the age of a food product.

In certain embodiments, a ratio between a rather stable and a rather unstable target molecule may by comprised in one element of the identifier. When compiling the identifier, the determined ratio between the rather stable and the rather unstable molecule may be compared to a threshold. In certain embodiments, the threshold may be defined based on a ratio that has been determined with a sample from a product that is known to be authentic. However, the threshold may also be adjusted based on the expected or known decay behavior of the rather stable and/or the rather unstable molecule. In certain embodiments, the threshold may also be defined based on the confidence interval that has been determined for the signal that corresponds to a rather stable molecule, a rather unstable molecule and/or a ratio between a rather stable and a rather unstable molecule.

### DETAILED DESCRIPTION OF THE INVENTION

The present invention shall, in particular, contribute to authenticate samples at a much more detailed level than so far possible.

The present invention provides a method for the identification of a product by correlating a set of specific (binding) molecules with a set of target molecules found in or derived from a sample of said product.

In particular, the invention provides a method for the authentication of (candidate) products based on the profile of selected nucleic acid sequences derived from the sample's microbiome and/or macrobiome, e.g. fungi, yeasts, bacteria and phages, as well as animal or plant species.

In particular, the method may comprise the steps of defining the genera or species to be identified for the sample authentication, selecting appropriate nucleic acid sequences for specific identification of the defined genera or species, isolation of the DNA or RNA from the sample, optional digestion and performing an amplification if required, identification and quantification of the specific sequences, and e.g. deriving an identifier in form of a digital code for each sample. The genera or species to be identified in such a method may be selected from the macrobiome or the microbiome or from both. Thereby, the microbiome and/or macrobiome of the product may be so specific that it can be differentiated from the microbiome and/or macrobiome of another product. Hence, a combination of selected representatives of the microbiome such as fungi, yeasts, bacteria, viruses, phages, archaea or protists may constitute a product-specific microbiome. In particular, specific bacteria genera or species may constitute such product-specific microbiome.

Then specific nucleic acid sequences of the product-specific microbiome or macrobiome may be selected. Preferably, these nucleic acid sequences belong to product-specific bacteria genera or species of the microbiome.

Also, DNA or RNA from the sample may be isolated according to procedures known to the person skilled in the art. For example, such DNA or RNA may be isolated with the help of silica (see CN101210032A) or by any other method known in the art or commercially available kit. After an optional digestion step, the DNA or RNA is amplified in order to obtain enough numbers of copies, if necessary.

Also, the specific sequences may be identified with the help of hybridization of complementary nucleic acid sequences and quantified based on a preselected threshold.

Also, a digital code may be a sequence of the figures zero and one, counted as presence or absence of a preselected specific nucleic acid sequence.

In one embodiment, the present invention relates to a method for providing an identifier for a product comprising the steps of:

a) obtaining a sample of the product;

b) contacting the sample with a set of molecules, particularly a set of binding molecules such as nucleic acid molecules, nanobodies, antibodies or antibody like polypeptides, or peptides, which are capable of recognizing and/or binding selected target molecules comprised in the sample, in particular comprised in members of the micro- and/or macrobiome comprised in the sample, such as nucleic acid molecules, peptides or small molecules preferably wherein target molecules in the sample are stable or unstable over time;

c) defining a specific determination threshold for each of the target molecules;

d) determining whether the target molecules are present in the sample, such that target molecules are considered present if their concentration and/or amount in the samples is equal to or above the determination threshold, and are considered absent if their concentration is below the determination threshold or such that target molecules are considered present if their concentration is within the determination range, and are considered absent if their concentration is outside of the determination range, respectively; it should be noted that the threshold may be well above the limit of detection and may also be well above the limit of quantification; in some instances, two different products both comprise a specific molecule, but in clearly different amounts; here, a threshold could be set such that the two products are differentiated when comparing the respective signal strengths. Also, certain ranges might be established so that the signal strength is compared against two thresholds;

e) obtaining an identifier for the product by correlating the molecules, particularly the binding molecules, used in b), with the presence or absence of the target molecules, particularly of the target molecules comprised in members of the micro- and/or macrobiome, in the sample.

In the present invention, binding molecules may comprise nucleic acid molecules, nanobodies, antibodies or antibody like polypeptides, or peptides. In case the binding molecules are nucleic acid molecules, such nucleic acid molecules may comprise specific nucleic acid sequences. The nucleic acid molecules or nucleic acid sequences may be partially that of a reference product to be compared. They may consist of DNA or RNA, preferably DNA. Preferably, the binding molecules are nucleic acid sequences, in particular ssDNA (single stranded DNA) sequences. The nucleic acid binding molecules are able to hybridize with complementary target DNA- or RNA-sequences. Such hybridized, i.e. double-stranded nucleic acid sequences may be detected by methods well known in the art, e.g. luminescence, fluorescence, potentiometric or amperometric systems.

In case the binding/recognition molecules are specific antibodies or antibody-like polypeptides or peptides, the binding to antigens (target molecules) in a sample is detected. The antigens may be peptides, carbohydrates or molecules of non-biological origin. The binding of the antibodies to their antigens may for instance be detected via ELISA (enzyme-linked immunosorbent assay) or multiplexed immunoassays and fluorescence tagged antibodies or antibody fragments, chemiluminescence or electrochemiluminescence, polarization assays, electrochemical signals or any kind of label free systems.

The target molecules comprised in the sample may be derived from the macrobiome or the microbiome of the product, preferably the microbiome. They may be selected from nucleic acid molecules, peptides or small molecules. Preferably they are nucleic acid molecules, preferably double stranded or single stranded DNA or RNA, in particular rRNA (ribosomal RNA). Preferred sequence lengths are up to 1000 nucleotides.

Depending on the product ageing, the microbiome may change. Therefore, specific microbiome component may be characteristic for a certain age of a product. Moreover, components of the macrobiome such as for instance DNA may decompose with age. Therefore, the particular length of DNA or RNA strands might be characteristic for a certain age of a product.

The definition of specific determination thresholds facilitates the evaluation whether a specific target molecule is considered as comprised in the sample or not.

If certain of the above steps are repeatedly executed for different products, a product matrix can be established that relates to all such products. In this product matrix, the identifier for every single product could be included, so that the product matrix is more precisely a product identifier matrix. The product identifier matrix may be obtained in the form of a digital code, i.e. a code which indicates the presence or absence of specific target molecules in the product sample.

Presence of such molecule may e.g. be indicated as “1”, absence may be indicated as “0”. The digital code would thus be a set of specific sequence of detectable binding molecules.

For instance, the set of molecules capable of recognizing and/or binding selected target molecules may be ordered such that binding molecule (1) is specific for bacterium A, (2) is a binding molecule specific for bacterium B, (3) is a binding molecule specific for bacterium C, (4) is a binding molecule specific for bacterium D, (5) is a binding molecule specific for bacterium E, (6) is a binding molecule specific for bacterium F. Note that the target molecules could be obtained during the analysis, e.g. by amplification. As the amplification process might produce a variety of molecules that while sharing the basic property of being detectable by the same binding molecule, they are still different to each other, it is obvious that the term “target molecule” is to be construed in a broad manner and should in particularly include a group of molecules obtained in an amplification process that all are detectable using the same binding molecules.

If the respective binding molecules are not capable of binding to their target molecules in the sample, this would mean that the target molecule is not present in the sample and that the corresponding signal strength of a detection signal would be below the threshold.

Thus, comparing signal strengths to the thresholds would lead to a digital code such as for instance 100111 in case of target molecules specific for bacteria A, D, E and F are present in the sample and target molecules specific for bacteria B and C are absent. From these codes obtained for N products, a matrix as shown in table 1 could be established. Note that the matrix shown in table 1 can be a matrix of products known to be genuine.

Now, as the candidate product of unknown origin is to be examined, the digital code corresponding to the candidate product is determined, and thereafter compared, for example via either an electronic automated process or by hand, to the digital code contained in the table 1 product identifier (6×N) matrix; as an alternative to comparing the candidate identifier code with each column in the matrix and outputting the result, the digital code corresponding to the identifier of the candidate product could be provided together with the matrix, for example extending the (6×N) matrix shown in table 1, by an additional column, so that a (6×N+1) matrix results. As may be seen from table 1, each product in the matrix can be differentiated from the other by its different digital code. The whole (6×N) matrix, however, can be considered an identifier for all genuine products selected for consideration. Note that where additional reference products are analyzed and the matrix is extended to include the additional reference products, it might become necessary to add one or more additional rows relating to one or more additional DNA sequences. Hence, where the database, reference library and hence the matrix is extended by M products using A additional sequences, the (6×N) matrix of table 1 would become a (6+A)×(N+M) matrix.

Table 1 demonstrates an example of a matrix of such embodiment:

| Binding molecule | Product 1 | Product 2 | . . . | Product N |
|---|---|---|---|---|
| 1) DNA Sequence A | 1 | 0 | | 0 |
| 2) DNA Sequence B | 0 | 1 | | 0 |
| 3) DNA Sequence C | 0 | 1 | | 1 |
| 4) DNA Sequence D | 1 | 1 | | 1 |
| 5) DNA Sequence E | 1 | 0 | | 0 |
| 6) DNA Sequence F | 1 | 1 | | 1 |
| Digital code | 100111 | 011101 | | 001101 |

Note that the six identifiers for the N products will be selected such that each digital code is by design different from every other digital code.

Also note that a binary 100111 could be transformed into the number 39, a digital code 011101 could be transformed into a number 13. Thus, the product code (identifier) for Product 1 could also be 39, or for Product 2 could be 13. However, where only a sparse data base is available, binary data may be highly preferred.

As may be seen from the above abstract example, each reference product can be differentiated from the other reference products by its digital code. The whole matrix, however, is providing reference identifier information relating to all reference products known to be genuine. It will be understood by the skilled person that a matrix is one form of storing and displaying reference information, so that the matrix can also be considered a library or database. However, for determining properties such as origin or vintage of a candidate product, a reference library or reference data base need not have the form of a matrix.

In case that the target molecules (or their precursors) are selected from the macrobiome, such as for instance a plant, one or more nucleic acid molecules, antibodies or antibody like polypeptides or peptides which are specific to the particular plant may be selected.

In case that the target molecules (or their precursors) are selected from the microbiome, such as for instance bacteria, one or more nucleic acid molecules, antibodies or antibody like polypeptides or peptides which are specific to the particular bacterium may be selected. Preferably, nucleic acid molecules are selected. The nucleic acid molecules may be specific to either a species or a genus of the bacteria. It is possible that a multiplicity of specific target molecules is characteristic for species of the same genus. It is also possible that a multiplicity of specific target molecules is characteristic for the same species.

In a more general embodiment of the invention, the product is foodstuff. In this context, the product may be non-processed, such as for instance wheat powder, or it may be processed.

In a preferred embodiment, the product is a processed product, in particular wine.

Wine is a highly processed product. The production process has undergone many stages such as fermentation, aging, clarification and filtration. The finished wine has very little DNA content, and the grape berry is introduced into the wine with a large amount of polyphenols (tannin). Complex chemical substances such as polysaccharides and organic acids seriously affect the quality of DNA extraction and downstream molecular biology analysis. While authentication of wine samples by DNA analysis may be based on the detection of specific DNA sequences of the grapes, environmental microorganisms that thus far were of interest only in respect of their impact on the flavor of the wine can also be referred to in authentication, identification and/or falsification of a wine. From this, it can be seen that the molecules considered to constitute part of a specific ensemble of molecules will be occurring naturally in the product. While they may stem from different sources such as grapes, yeast, the wood barrel, cork and the like, they need not to be added in an additional step for the purpose of identification.

Thus, even though it is possible to extract DNA from grapes contained in the wine for the method of the present invention, there is also the possibility to select markers which are specific to the microbiome of a certain wine.

DNA amplification by PCR is susceptible to inhibition by certain compounds in the wine. DNA stability is an issue, particularly as the DNA of microorganisms active in the early stage of the fermentation process might be degraded.

Both aspects, instability and inhibition, are detrimental for the reproducible detection and quantification of DNA sequences and subsequent library based authentication.

The present invention proposes in a preferred embodiment to define a set of DNA sequences from both plant and at least one of fungi, bacteria and phages potentially present in wine. Whereas the plant DNA from the grapevine is assumed to be indicative for the uniformity of the grape content in the wine, assumed not to undergo significant changes from production cycle to production cycle, environmental microbial, fungal or viral DNA have been found to vary from production cycle to production cycle.

In a further embodiment of the present invention, at least some of the target molecules or markers are thus selected from the microbiome of the product, for instance from the microbiome of the wine. Such microbiome may comprise fungi, yeasts, bacteria, viruses, phages, archaea and/or protists, preferably fungi, yeasts, bacteria and/or phages.

A data set comprising DNA sequences of grape varieties, typical microorganisms, fungi, and/or pathogens potentially present in wine may be defined. One or more DNA sequences of each species are selected to detect the presence of said species with a high sensitivity and selectivity.

As can be considered from the above, the presence of species may be defined by comparison to experimentally determined threshold values, and a minimum of sequences of the species that need to be equal to or above this threshold value.

Based on the presence or absence of a species, the identifier for each wine is derived, and consequently, a product identifier matrix or database of information relating to genuine products can be established. However, it will be understood that while such an identifier matrix is a good visualization, the information contained therein might also be used in the form of a data base or the like.

For instance, an identifier matrix may be composed of a two-dimensional display or a table wherein each of the columns represents a product, and each of the rows represent a target molecule or origin of a target molecule. Each of the products is then characterized by the specific digital code, indicating i.e. the presence or absence of said target molecule and/or the amount thereof. Where a simple comparison against a threshold is made, each identifier matrix element may be either “true” or “false”, depending on whether or not the signal relating to the respective target molecule is strong enough to exceed a useful threshold during analysis. Also, the indicator could have been compared against one of several ranges and its value could be e.g. “high”, “medium” or “low”. Note that in particular, where a database is to be established, it might be useful to use non-binary identifier elements, that is, multistep digitized values so that when later on adding additional references, the thresholds can be more easily adjusted as necessary. It will be obvious that instead of using a “High” “medium” or “low” indication for certain identifier elements, it would be possible to compare a given signal strength against a first threshold, treat the respective result of this comparison as a first identifier element, to then compare the same signal strength against another threshold and treat the respective result as a second comparison result.

Note that in some instances, a signal relating to a given target molecule and very strong in other genuine products could yield a very weak signal for a particular other genuine product. For this other genuine product, if the signal is so weak that is hardly observable, but can be expected to vanish in the near future due to an instability of the target molecule in the product, it might be useful to set a threshold such that the target molecule is considered to be NONdetectable.

To overcome the DNA quantification issues such as degradation of DNA, depurination, PCR inhibitors present in wine and so forth, the authentication is based on a threshold applied to the detectability of DNA sequences. The authentication is therefore less dependent on the accuracy and precision of the amount of sequences present in the wine sample.

Table 2 shows an example for markers from the microbiome of wine:

Producer

| | A | B |
|---|---|---|
| Year | 2016 | 2013 |
| Acetobacter | 0 | 1 |
| Acinetobacter | 1 | 0 |
| Arsenophonus | 1 | 1 |
| Bacillus | 1 | 0 |
| Brevibacillus | 1 | 0 |
| Burkholderia | 0 | 0 |
| Dyella | 0 | 0 |
| Oenococcus | 0 | 1 |
| Pelomonas | 0 | 0 |
| Salinispora | 0 | 1 |
| Streptococcus | 0 | 0 |
| Tanticharoenia | 0 | 0 |
| Control Marker | 1 | 1 |

In Table 2, the code for producer A year 2016 reads: 0111100000001 whereas the one for producer B year 2013 reads: 1010000101001. The number N of bits in the code, that is, the N-bit resolution of the product identifier matrix, is defined so as to allow for a proper identification of all wines to be analyzed.

For a better understanding, Acetobacter is a genus of acetic acid bacteria. Acetic acid bacteria are characterized by the ability to convert ethanol to acetic acid in the presence of oxygen. Of these, the genus Acetobacter is distinguished by the ability to oxidize lactate and acetate into carbon dioxide and water (Cleenwerck I; Vandemeulebroecke D; Janssens D; Swings J (2002), “Re-examination of the genus Acetobacter, with descriptions of Acetobacter cerevisiae sp. nov. and Acetobacter malorum sp. nov”, International Journal of Systematic and Evolutionary Microbiology. 52: 1551-1558). Bacteria of the genus Acetobacter have been isolated from industrial vinegar fermentation processes and are frequently used as fermentation starter cultures (Sokollek S J; Hertel C; Hammes W P (February 1998), “Cultivation and preservation of vinegar bacteria”, Journal of Biotechnology. 60 (3): 195-206).

An exemplary nucleotide sequence comprised in bacteria belonging to the genus Acetobacter is shown in SEQ ID NO:1. Thus, in one embodiment, a target molecule in the methods of the present invention is SEQ ID NO:1. Accordingly, the set of molecules used in the methods of the present invention may comprise one or more molecules targeting SEQ ID NO:1.

CCTACGGGTGGCTGCAGTGGGGAATTTTGGACAATGGGCGAAAGCCTGA
TCCAGCAATGCCGCGTGTGTGAGAGTAACTGCTCATGCAGTGACGGTAT
CCAACCAGAAAGCCCCGGCTAACTTCGTGCCAGCAGCCGCGGTAATACG
AAGGGGGCTAGCGTTGCTCGGAATGACTGGGCGTAAAGGGCGTGTAGGC
GGTTTGTACAGTCAGATGTGAAATCCCCGGGCTTAACCTGGGAGCTGCA
TTTGATACGTGCAGACTAGAGTGTGAGAGAGGGTTGTGGAATTCCCAGT
GTAGAGGTGAAATTCGTAGATATTCGGAAGAACACCAGTGGCGAAGGCG
ACTCGCTGGACAAGTATTGACGCTGAGGTGCGAAAGCGTGGGGAGCAAA
CAGGATTAGATACCCCGGTAGTC

Acinetobacter is a genus of gram-negative, non-fermenting bacteria that belong in the family Moraxellaceae. An exemplary nucleotide sequence comprised in bacteria belonging to the genus Acinetobacter is shown in SEQ ID NO:2. Thus, in one embodiment, a target molecule in the methods of the present invention is SEQ ID NO:2. Accordingly, the set of molecules used in the methods of the present invention may comprise one or more molecules targeting SEQ ID NO:2.

CCTACGGGAGGCAGCAGTGGGGAATATTGGACAATGGGGGGAACCCTGA
TCCAGCCATGCCGCGTGTGTGAAGAAGGCCTTTTGGTTGTAAAGCACTT
TAAGTTGGGAGGAAGGGCATTAACCTAATACGTTAGTGTTTTGACGTTA
CCGACAGAATAAGCACCGGCTAACTCTGTGCCAGCAGCCGCGGTAATAC
AGAGGGTGCAAGCGTTAATCGGATTTACTGGGCGTAAAGCGCGCGTAGG
TGGCCAATTAAGTCAAATGTGAAATCCCCGAGCTTAACTTGGGAATTGC
ATTCGATACTGGTTGGCTAGAGTATGGGAGAGGATGGTAGAATTCCAGG
TGTAGCGGTGAAATGCGTAGAGATCTGGAGGAATACCGATGGCGAAGGC
AGCCATCTGGCCTAATACTGACACTGAGGTGCGAAAGCATGGGGAGCAA
ACAGGATTAGATACCCGAGTAGTC

Bacillus is a genus of gram-positive, rod-shaped bacteria which comprise more than 200 species. Characteristic of the genus Bacillus is the formation of endospores and aerobic or facultative aerobic growth. Some species can be pathogenic. An exemplary nucleotide sequence comprised in bacteria belonging to the genus Bacillus is shown in SEQ ID NO:3. Thus, in one embodiment, a target molecule in the methods of the present invention is SEQ ID NO:3. Accordingly, the set of molecules used in the methods of the present invention may comprise one or more molecules targeting SEQ ID NO:3.

CCTACGGGAGGCAGCAGTAGGGAATCTTCCGCAATGGACGAAAGTCTGA
CGGAGCAACGTCGCGTGAGTGATGAAGGCTTTCGGGTCGTAAAACTCTG
TTGTTAGGGAAGAACAAGTGCTAGTTGAATAAGCTGGCACCTTGACGGT
ACCTAACCAGAAAGCCACGGCTAACTACGTGCCAGCAGCCGCGGTAATA
CAGAGGGTGCGAGCGTTAATCGGATTTACTGGGCGTAAAGCGCGCGCAG
GCGGCTATGTAAGTCTGGTGTTAAAGCCCGGAGCTCAACTCCGGTTCGC
ATCGGAAACTGTGTAGCTTGAGTGCAGAAGAGGAAAGCGGTATTCCACG
TGTAGCGGTGAAATGCGTAGAGATGTGGAGGAACACCAGTGGCGAAGGC
GGCTTTCTGGTCTGTAACTGACACTGAGGCGCGAAAGCGTGGGGAGCAA
ACAGGATTAGATACCCGAGTAGTC

Brevibacillus is a genus of Gram-positive bacteria in the family Paenibacillaceae (Shida, O.; Takagi, H.; Kadowaki, K.; Komagata, K. (October 1996), “Proposal for two new genera, Brevibacillus gen. nov. and Aneurinibacillus gen. nov”, International Journal of Systematic Bacteriology. 46 (4): 939-946). An exemplary nucleotide sequence comprised in bacteria belonging to the genus Brevibacillus is shown in SEQ ID NO:4. Thus, in one embodiment, a target molecule in the methods of the present invention is SEQ ID NO:4. Accordingly, the set of molecules used in the methods of the present invention may comprise one or more molecules targeting SEQ ID NO:4.

CCTACGGGGGGCTGCAGTAGGGAATTTTCCACAATGGACGAAAGTCTGA
TGGAGCAACGCCGCGTGAACGATGAAGGTCTTCGGATTGTAAAGTTCTG
TTGTCAGGGATGAACACGTACCGTTCGAATAGGGCGGTACCTTGACGGT
ACCTGACGAGAAAGCCACGGCTAACTACGTGCCAGCAGCCGCGGTAATA
CGTAGGTGGCAAGCGTTGTCCGGATTTATTGGGCGTAAAGCGCGCGCAG
GCGGCTATGTAAGTCTGGTGTTAAAGCCCGGAGCTCAACTCCGGTTCGC
ATCGGAAACTGTGTAGCTTGAGTCTCGTAGAGGGGGGTAGAATTCCAGG
TGTAGCGGTGAAATGCGTAGAGATCTGGAGGAATACCGGTGGCGAAGGC
GGCCCCCTGGACGAAGACTGACGCTCAGGTGCGAAAGCGTGGGGAGCAA
ACAGGATTAGATACCCCAGTAGTC

The Burkholderia (previously part of Pseudomonas) genus name refers to a group of virtually ubiquitous Gram-negative, obligately aerobic, rod-shaped bacteria that are motile by means of single or multiple polar flagella, with the exception of Burkholderia mallei which is nonmotile. Members belonging to the genus do not produce sheaths or prosthecae and are able to utilize poly-beta-hydroxybutyrate (PHB) for growth. The genus includes both animal and plant pathogens, as well as some environmentally important species. An exemplary nucleotide sequence comprised in bacteria belonging to the genus Burkholderia is shown in SEQ ID NO:5. Thus, in one embodiment, a target molecule in the methods of the present invention is SEQ ID NO:5. Accordingly, the set of molecules used in the methods of the present invention may comprise one or more molecules targeting SEQ ID NO:5.

CCTACGGGGGGCAGCAGTGGGGAATATTGGACAATGGGCGCAAGCCTGA
TCCAGCAATGCCGCGTGTGTGAAGAAGGTCTTCGGATTGTAAAGCGCTT
TTGTCCGGGAAGAAATCCTTTCTGATAATACCGGAGGGGGATGACGGTA
CCGGAAGAAGAAGCCCCGGCTAACTTCGTGCCAGCAGCCGCGGTAATAC
GTAGGGTGCGAGCGTTAATCGGAATTACTGGGCGTAAAGCGTGCGCAGG
CGGTGATGTAAGACCGATGTGAAATCCCCGGGCTTAACCTGGGAACTGC
ATTGGTGACTGCATCGCTGGAGTATGGCAGAGGGGGGTGGAATTCCACG
TGTAGCAGTGAAATGCGTAGAGATGTGGAGGAACACCGATGGCGAAGGC
AGCCCCCTGGGCCAATACTGACGCTCATGCACGAAAGCGTGGGGAGCAA
ACAGGATTAGATACCCGGGTAGTC

Dyella is a genus of Proteobacteria from the family of Rhodanobacteraceae. An exemplary nucleotide sequence comprised in bacteria belonging to the genus Dyella is shown in SEQ ID NO:6. Thus, in one embodiment, a target molecule in the methods of the present invention is SEQ ID NO:6. Accordingly, the set of molecules used in the methods of the present invention may comprise one or more molecules targeting SEQ ID NO:6.

CCTACGGGAGGCTGCAGTGGGGAATATTGGACAATGGGCGCAAGCCTGA
TCCAGCAATGCCGCGTGTGTGAAGAAGGTCTTCGGATTGTAAAGCACTT
TCGACGGGGACGATGATGACGGTACCCGTAGAAGAAGCCCCGGCTAACT
TCGTGCCAGCAGCCGCGGTAATACGAAGGGGGCTAGCGTTGCTCGGAAT
GACTGGGCGTAAAGCGTGCGCAGGCGGTGATGTAAGACCGATGCGAAAT
CCCCGGGCTTAACCTGGGAATGGCAGTGGATACTGGATCGCTAGAGTGT
GATAGAGGATGGTGGAATTCCCGGTGTAGCGGTGAAATGCGTAGAGATC
GGGAGGAACATCAGTGGCGAAGGCGGCCATCTGGATCAACACTGACGCT
GAGGCACGAAAGCGTGGGGAGCAAACAGGATTAGATACCCCAGTAGTC

Oenococcus is a genus of Gram-positive bacteria, placed within the family Leuconostocaceae. The only species in the genus was Oenococcus oeni (which was known as Leuconostoc oeni until 1995). In 2006, the species Oenococcus kitaharae was identified. As its name implies, Oenococcus oeni holds major importance in the field of oenology, where it is the primary bacterium involved in completing the malolactic fermentation (Kunkee, R. E. 1973. Malo-Lactic Fermentation and Winemaking. In, The Chemistry of Winemaking, Adv. Chem. Ser. 137, A. D. Webb, Ed. American Chemical Society. Washington D.C.). An exemplary nucleotide sequence comprised in bacteria belonging to the genus Oenococcus is shown in SEQ ID NO:7. Thus, in one embodiment, a target molecule in the methods of the present invention is SEQ ID NO:7. Accordingly, the set of molecules used in the methods of the present invention may comprise one or more molecules targeting SEQ ID NO:7.

CCTACGGGCGGCAGCAGTGGGGAATATTGGACAATGGGCGCAAGCCTGA
TCCAGCAATGCCGCGTGTGTGATGAAGGCTTTCGGGTCGTAAAGCACTG
TTGTAAGGGAAGAATAACTGAATTCAGAGAAAGTTTTCAGCTTGACGGT
ACCTTACCAGAAAGGGATGGCTAAATACGTGCCAGCAGCCGCGGTAATA
CGTATGTCCCGAGCGTTATCCGGATTTATTGGGCGTAAAGCGAGCGCAG
ACGGTTTATTAAGTCTGATGTGAAATCCCGAGGCCCAACCTCGGAACTG
CATTGGAAACTGATTTACTTGAGTGCGATAGAGGCAAGTGGAACTCCAT
GTGTAGCGGTGAAATGCGTAGATATCTGGAAGAACACCGATGGCGAAGG
CAACCTCCTGGGCCTGTTCTGACGCTGAGGCACGAAAGCGTGGGTAGCA
AACAGGATTAGATACCCCAGTAGTC

Pelomonas is a genus of Gram-negative, rod-shaped, nonspore-forming bacteria from the family Comamonadaceae. An exemplary nucleotide sequence comprised in bacteria belonging to the genus Pelomonas is shown in SEQ ID NO:8. Thus, in one embodiment, a target molecule in the methods of the present invention is SEQ ID NO:8. Accordingly, the set of molecules used in the methods of the present invention may comprise one or more molecules targeting SEQ ID NO:8.

CCTACGGGTGGCTGCAGTGGGGAATTTTGGACAATGGGCGCAAGCCTGA
TCCAGCCATGCCGCGTGCGGGAAGAAGGCCTTCGGGTTGTAAACCGCTT
TTGTCAGGGAAGAAAAGGTTCTGGTTAATACCTGGGACTCATGACGGTA
CCTGAAGAATAAGCACCGGCTAACTCCGTGCCAGCAGCCGCGGTAATAC
GGAGGGTGCAAGCGTTAATCGGAATTACTGGGCGTAAAGCGCACGCAGG
CGGTTTGTTAAGTCAGATGTGAAATCCCCGGGCTCAACCTGGGAACTGC
ATCTGATACTGGCAAGCTTGAGTCTCGTAGAGGGGGGTAGAATTCCAGG
TGTAGCGGTGAAATGCGTAGAGATCTGGAGGAATACCGATGGCGAAGGC
AGGTTACTGGGCAGTTACTGACGCTGAGGAGCGAAAGCATGGGTAGCAA
ACAGGATTAGATACCCTGGTAGTC

Salinispora is genus of bacteria which belong to family of Micromonosporaceae. An exemplary nucleotide sequence comprised in bacteria belonging to the genus Salinispora is shown in SEQ ID NO:9. Thus, in one embodiment, a target molecule in the methods of the present invention is SEQ ID NO:9. Accordingly, the set of molecules used in the methods of the present invention may comprise one or more molecules targeting SEQ ID NO:9.

CCTACGGGGGGCTGCAGTGGGGAATTTTGGACAATGGGGGAAACCCTGA
TCCAGCAATGCCGCGTGTGTGAAGAAGGCCTTCGGGTTGTAAAGCACTT
TTGTCCGGGAAGAAATCCTTTCTGATAATACTGGAGGGGGATGACGGTA
CCGGAAGAATAAGCACCGGCTAACTACGTGCCAGCAGCCGCGGTAATAC
GCAGGGTGCGAGCGTTAATCGGAATTACTGGGCGTAAAGCGTGCGCAGG
CGGTGATGTAAGACCGATGTGAAATCCCCGGGCTTAACCTGGGAACTGC
ATTGGTGACTGCATTGCTGGAGTATGGCAGAGGGGGGTGGAATTCCACG
TGTAGCAGTGAAATGCGTAGAGATGTGGAGGAACACCGATGGCGAAGGC
AGCCCCCTGGGCCAATACTGACGCTCATGCACGAAAGCGTGGGGAGCAA
ACAGGATTAGATACCCGTGTAATC

Streptococcus is a genus of gram-positive coccus (plural cocci), or spherical bacteria, that belongs to the family Streptococcaceae, within the order Lactobacillales (lactic acid bacteria), in the phylum Firmicutes (Ryan K J, Ray C G, eds. (2004). Sherris Medical Microbiology (4th ed.), McGraw Hill. pp. 293-4, ISBN 0-8385-8529-9). An exemplary nucleotide sequence comprised in bacteria belonging to the genus Streptococcus is shown in SEQ ID NO:10. Thus, in one embodiment, a target molecule in the methods of the present invention is SEQ ID NO:10. Accordingly, the set of molecules used in the methods of the present invention may comprise one or more molecules targeting SEQ ID NO:10.

CCTACGGGGGGCTGCAGTAGGGAATCTTCGGCAATGGGGGGAACCCTGA
CCGAGCAACGCCGCGTGAGTGAAGAAGGTTTTCGGATCGTAAAGCTCTG
TTGTAAGAGAAGAACGGGTGTGAGAGTGGAAAGTTCACACTGTGACGGT
ATCTTACCAGAAAGGGACGGCTAACTACGTGCCAGCAGCCGCGGTAATA
CGTAGGTCCCGAGCGTTGTCCGGATTTATTGGGCGTAAAGCGAGCGCAG
GCGGTTAGATAAGTCTGAAGTTAAAGGCTGTGGCTTAACCATAGTATGC
TTTGGAAACTGTTTAACTTGAGTGCAGAAGGGGAGAGTGGAATTCCATG
TGTAGCGGTGAAATGCGTAGATATATGGAGGAACACCAGTGGCGAAGGC
GACTTTCTGGTCTGTAACTGACACTGAGGCGCGAAAGCGTGGGGAGCAA
ACAGGATTAGATACCCTGGTAGTC

Arsenophonus is a genus of Enterobacteriaceae, of the Gammaproteobacteria (Gherna, Robert L., et al. “NOTES: Arsenophonus nasoniae gen. nov., sp. nov., the Causative Agent of the Son-Killer Trait in the Parasitic Wasp Nasonia vitripennis.” International Journal of Systematic Bacteriology 41.4 (1991): 563-565). As the marker relating to Arsenophonus is “1” for both wines considered, it does not help to differentiate between the two wines of producer A and B. Hence, no sequence is indicated.

Tanticharoenia is a genus in the family of Acetobacteraceae. As the marker relating to Arsenophus is “0” for both wines considered, it does not help to differentiate between the two wines of producer A and B. Hence, no sequence is indicated.

In a further embodiment of the present invention, the (or some) target molecules or markers are selected from the macrobiome, which comprises plants, and in particular vine. It is assumed that plant DNA, i.e. DNA from the grapes, does not undergo significant changes from one production cycle to the other. It can therefore be the target in case the grape composition of the wine needs to be examined. Note that any target molecule or marker selected from the macrobiome may either be contained in the macrobiome itself or be derived therefrom, e.g. during storage and/or analysis. The same of course holds for target molecule or marker selected from the microbiome.

In a further embodiment of the invention, one or multiple sets of molecules capable of recognizing and/or binding selected target or marker molecules, in particular nucleic acid molecules, antibodies or antibody like polypeptides, or peptides are provided which are specific for genera, preferably species, comprised in the macro- and/or microbiome comprised in the sample. Preferably, the target molecules are nucleic acid molecules. Preferably, the nucleic acid molecules are comprised in the microbiome of the product. Even more preferably, the nucleic acid molecules are comprised in fungi or bacteria of the microbiome, in particular in bacteria. Such bacteria are for instance the bacteria of Table 2.

In a further embodiment of the invention, the set of different distinguishable molecules comprises at least one nucleic acid molecule, and a step is used comprising the use of hybridization of nucleic acid molecules to complementary sequences on a microarray, PCR amplification methods and/or sequencing, in particular next generation sequencing.

Preferably, said PCR amplification method is multiplex real-time PCR. Real-time multiplex PCR is able to detect, differentiate, and provide a quantitative result for many different targets.

In a preferred embodiment of the invention, said at least one nucleic acid molecule targets the bacterial 16S rRNA genes, for instance the 16S rRNA gene of the bacteria listed in Table 3.

In a further embodiment of the invention, the set of different distinguishable molecules comprises at least one antibody or antibody-like polypeptide and step c) comprises the use of immuno assay methods in a sandwich or competitive format.

Preferably, the immuno assay method comprises the use of a tracer antibody, antibody fragment or antibody-like polypeptide for detection.

A further embodiment of the invention relates to a method for determining the origin of a candidate product, wherein the origin of the product is that of a reference or of a product known to be genuine. This means that in case the identifier matches the identifier of the reference or genuine product, the candidate product may be considered an original product. However, as stated above, the match need not be perfect. In certain cases, an alleged origin of a candidate product can be considered to have been verified even despite a non-perfect match to a reference product identifier, for example where a very large number of non-binary identifier elements have been provided and some of the non-binary reference identifier elements show some discrepancy to the corresponding candidate identifier elements.

A further embodiment of the invention relates to the determination of the age of a processed product, wherein the age of the processed product is that of the reference product. Such an embodiment may use binding/recognition molecules which are nucleic acid molecules that target bacterial 16S rRNA genes of the samples. A particularly preferred embodiment may relate to the determination of the age of the product by relating to the vintage of the product where the product is a wine.

To overcome potential quantification issues due to degradation of the analytes during the storage of the food stuff or wine, an algorithm is developed reflecting the analyte changes during storage, which allows to predict the content of analytes after long storage periods. For example, the product might be subjected to an oxidation process that effectively changes the amount and/or structure of target molecules (or their precursors). In case of a wine bottle, such an oxidation process will come to a steady state once the initial oxygen supply in the bottle is depleted and further oxygen is only available by diffusion through the cork. Of course, if this is the case, defects in the cork or an initial difference in the filling level may result in a depletion that differs from bottle to bottle.

Moreover, a further embodiment of the invention relates to the authentication of a processed product, wherein the processed product is determined to be authentic if origin and/or age are identical to the labeling of the processed product.

Another embodiment of the invention relates to the use of the methods as described herein for the identification of the origin and/or the age of a product.

A further embodiment of the invention relates to a device for performing the inventive method as disclosed herein.

Another embodiment of the present invention relates to a kit for performing the inventive method as disclosed herein.

### EXAMPLES

### Example 1

Various Bourgogne wines from different regions were investigated for the presence of distinct bacterial species. To do so, the inventors applied a DNA extraction method on the wine sample followed by the amplification of the bacterial 16S rRNA gene using primers designed at conserved regions of the gene. The amplicon was then subjected to Illumine New Generation DNA sequencing and the obtained sequences were meta-genomically analyzed and annotated. The results are summarized in the below table and they demonstrate that each of the investigated wines shows a distinct pattern of bacterial DNA sequences that can be expressed digitally (Table 3).

Again, note that for the example given, the markers relating to Tanticharoenia and Arsenophonus are not necessary to differentiate the wines listed.

TABLE 3
Patterns of bacterial DNA sequences in different wine samples

| | Producer | | | | | | | |
|---|---|---|---|---|---|---|---|---|
| | Aide | | | | | | | |
| | Gott | Durbacher | Durbacher | Zlereisen | Zlereisen | Zlereisen | C. Picon | C. Picon |
| | Year | | | | | | | |
| Organisms (Genus) | 2016 | 2013 | 2014 | 2011 | 2013 | 2014 | 1969 | 1986 |
| Acetobacter | 0 | 1 | 0 | 1 | 1 | 1 | 0 | 1 |
| Acinetobacter | 1 | 0 | 0 | 0 | 0 | 0 | 1 | 0 |
| Arsenophonus | 1 | 1 | 1 | 0 | 0 | 0 | 0 | 0 |
| Bacillus | 1 | 0 | 0 | 0 | 0 | 0 | 0 | 0 |
| Brevibacillus | 1 | 0 | 0 | 0 | 0 | 0 | 0 | 0 |
| Burkholderia | 0 | 0 | 0 | 0 | 0 | 1 | 0 | 0 |
| Dyella | 0 | 0 | 0 | 0 | 0 | 0 | 1 | 1 |
| Oenococcus | 0 | 1 | 0 | 0 | 1 | 1 | 0 | 0 |
| Pelomonas | 0 | 0 | 0 | 0 | 0 | 0 | 0 | 0 |
| Salinispora | 0 | 1 | 0 | 1 | 1 | 1 | 1 | 1 |
| Streptococcus | 0 | 0 | 0 | 0 | 0 | 0 | 0 | 1 |
| Tanticharoenia | 0 | 0 | 0 | 1 | 1 | 1 | 0 | 0 |
| Control Marker | 1 | 1 | 1 | 1 | 1 | 1 | 1 | 1 |

### Example 2: Identification and Classification of DNA from Wine Samples

Material and Methods

Material

A list of the kits, reagents and equipment used for DNA extraction, library generation and sequencing are summarized in following tables.

TABLE 4
Nucleic acid extraction, purification and quantification reagents

| Kit | Application | Catalog nr. | Supplier |
|---|---|---|---|
| Qubit 1x dsDNA HS | Nucleic acid | Q33231 | ThermoFisher |
| Assay | quantification | | |
| DNeasy maricon Food | Nucleic acid | 69514 | QIAGEN |
| (50) | extraction | | |
| AMPure XP | Nucleic acid | A63880 | Beckman |
| | purification | | Coulter |

TABLE 5
Library preparation and sequencing reagents

| Kit | Application | Catalog nr. | Supplier |
|---|---|---|---|
| REPLI-g Sigle Cell | Nucleic acid pre | 150343 | QIAGEN |
| WGA (24) | amplification | | |
| KAPA HiFi HotStart | 16S library | 07958927001 | Roche |
| Ready Mix | generation | | |
| Nextera ™ DNA CD | Whole-genome | 20015881 | Illumina |
| Index Primer | library generation | | |
| Nextera ™ DNA Flex | Whole-genome | 20018704 | Illumina |
| Library Prep | library generation | | |
| PhiX Control v3 | Internal control | FC-110-3001 | Illumina |
| | for sequencing | | |
| MiSeq Reagent Kit v2 | Sequencing | MS-102-2002 | Illumina |
| Mikro 300 cylces | cartridge | | |
| MiSeq v2 Reagent Kit | Sequencing | MS-103-1003 | Illumina |
| 500 cycles PE | cartridge | | |

TABLE 6
Primer used for library generation

| SEQ | | |
|---|---|---|
| ID | | |
| NO | Primer | Sequence (5′-3′) |
| 11 | V3 | TCGTCGGCAGCGTCAGATGTGTATAAGAGA |
| | | CAGCCTACGGGNGGCWGCAG |
| 12 | V4 | GTCTCGTGGGCTCGGAGATGTGTATAAGAG |
| | | ACAGGACTACHVGGGTATCTAATCC |
| 13 | NGS_i5_S518 | AATGATACGGCGACCACCGAGATCTACACC |
| | | TATTAAGTCGTCGGCAGCGTC |
| 14 | NGS_i5_S520 | AATGATACGGCGACCACCGAGATCTACACA |
| | | AGGCTATTCGTCGGCAGCGTC |
| 15 | NGS_i5_S521 | AATGATACGGCGACCACCGAGATCTACACG |
| | | AGCCTTATCGTCGGCAGCGTC |
| 16 | NGS_i7_N720 | CAAGCAGAAGACGGCATACGAGATAGGCTC |
| | | CGGTCTCGTGGGCTCGG |
| 17 | NGS_i7_N721 | CAAGCAGAAGACGGCATACGAGATGCAGCG |
| | | TAGTCTCGTGGGCTCGG |
| 18 | NGS_i7_N722 | CAAGCAGAAGACGGCATACGAGATCTGCGC |
| | | ATGTCTCGTGGGCTCGG |
| 19 | NGS_i7_N723 | CAAGCAGAAGACGGCATACGAGATGAGCGC |
| | | TAGTCTCGTGGGCTCGG |
| 20 | NGS_i7_N724 | CAAGCAGAAGACGGCATACGAGATCGCTCA |
| | | GTGTCTCGTGGGCTCGG |
| 21 | NGS_i7_N726 | CAAGCAGAAGACGGCATACGAGATGTCTTA |
| | | GGGTCTCGTGGGCTCGG |
| 22 | NGS_i7_N727 | CAAGCAGAAGACGGCATACGAGATACTGAT |
| | | CGGTCTCGTGGGCTCGG |
| 23 | NGS_i7_N728 | CAAGCAGAAGACGGCATACGAGATTAGCTG |
| | | CAGTCTCGTGGGCTCGG |

Primer sequences were developed by Illumina.

V3, V4 primer were synthetized by Microsynth AG, whereas i5/i7 primer were purchased from Illumina

TABLE 7
Equipment

| Equipment | Supplier |
|---|---|
| MiSeq | Illumina |
| Qubit ™ Fluorometer | ThermoFisher |
| Termocycler | Biometra |

Methods

DNA Extraction and Pre-Amplification

For DNA extraction, an appropriate volume of wine was aspired through the cork using a Coravind device and centrifuged at maximal speed for 10 minutes. Supernatant was discarded and pellet was subjected to extraction and purification using DNeasy mericon Food kit (Qiagen) according to the manufacturer's instructions. Volume of chloroform as well as buffer used in subsequent steps were adjusted to achieve maximal DNA recovery. Elution was performed in two steps using the appropriate volume of elution buffer to reach the best dilution to yield ratio. DNA was either stored at −20° C. or directly processed.

Prior to library preparation, the isolated genomic DNA was pre-amplified using the REPLI-g Single Cell WGA kit (Qiagen), according to the manufacturer's protocol. Reactions were incubated at 30° C. for different times, depending on downstream application.

Library Generation and Sequencing

16S rRNA Library Generation

Pre-amplified DNA was diluted with nuclease free water (either 1:100 or 1:200) and amplified using primer V3 and V4 (Table 6) aiming at the hypervariable region of 16S rRNA gene. Reactions were carried out in 25 μl, containing 0.2 μM of each primer and 1× KAPA HiFi HotStart Ready Mix (Roche). PCR consisted of a denaturation step at 95° C. for 3 min, followed by 25 or 35 cycles of 95° C. for 20 sec, 55° C. for 30 sec and 72° C. for 40 sec. A final denaturation step of 5 minutes at 72° C. was performed at the end of the cycles. In order to generate amplicons containing unique i5/i7 index combinations (Nextera™ DNA CD Index, Illumina) and to enable pooling of several samples, amplicons obtained with the V3/V4 primer pair were subjected to another PCR. Reactions were carried out in 25-50 μl, containing 0.2 μM of a unique primer combination and 1× KAPA HiFi HotStart Ready Mix (Roche). PCR consisted of a denaturation step at 95° C. for 2 min, followed by 15 cycles of 98° C. for 20 sec, 55° C. for 30 sec and 72° C. for 40 sec. A final denaturation step of 5 minutes at 72° C. was performed at the end of the cycles.

Prior to sample dilution and library pooling, products were cleaned up using magnetic beads, AMPure XP (Beckman Coulter) and eluted in nuclease free elution buffer. Concentration was assessed on a Qbit spectrophotometer using Qubit 1× dsDNA HS Assay. Samples were diluted to 4 mM and pooled for library denaturation.

Whole-Genome Library Generation

Pre-amplified DNA was diluted with nuclease free water (either 1:100 or 1:200) and 100 to 500 ng were used for fragmentation and adaptor ligation according to the manufacturer's protocol (Nextera™ DNA Flex Library Prep, Illumina). In order to generate fragments containing unique index combination (Nextera™ DNA CD Index, Illumina) and enable pooling of several samples, adaptor-ligated fragments were subjected to PCR, as described in the manufacturer's protocol (Nextera™ DNA Flex Library Prep, Illumina). Prior to sample dilution and library pooling, products were cleaned up using magnetic beads, AMPure XP (Beckman Coulter) and eluted in nuclease free elution buffer. Concentration was assessed on a Qbit spectrophotometer using Qubit 1× dsDNA HS Assay. Samples were diluted to 4 mM and pooled for library denaturation.

Library Denaturation and Sequencing

For paired end sequencing, a MiSeq from Illumina was used. 16S or whole-genomic pooled libraries containing 1 to 5% PhiX control v3 DNA were desaturated using freshly prepared NaOH (0.2 N) and diluted with HT buffer according to correspondent protocols (i.e. 16S metagenomic and Nextera Flex library, respectively). 6 pM of 16S library or 10 pM of whole-genomic library were loaded on a MiSeq v2 nano (500 cycles) or MiSeq v2 Mikro (300 cycles) cartridge, respectively. Cluster densities varied between 600 and 800 K/mm² for MiSeq v2 nano and between 800 and 1100 K/mm² for MiSeq v2 Mikro cartridges.

Data Analysis

Fastq files were analysed on SequenceHub platform (Illumina) using 16S metagenomic and kraken workflows for 16S and whole-genome analysis, respectively. A detection limit of 100 reads was set and only organisms above this value were considered positive.

Results

Several bacteria and eukaryotic organisms (using whole-genome sequencing) were detected in different wines. Based on these preliminary studies, a wine specific fingerprint can be generated.

A list of the representing detected organisms (bacteria, plant and fungi) including taxid and DNA sequence is shown in Table 8:

TABLE 8
Organisms identified in wine samples

| Num- | | | |
|---|---|---|---|
| ber of | | | |
| reads | Wine detection | TaxID | Species |
| 7408 | Chateau Leoville, 2002 | 326968 | Ziziphus jujuba |
| 675 | Rioja Crianza, 2015 | 343 | Xanthomonas |
| | | | translucens |
| 140 | Primitivo del Salento, 2015 | 90270 | Xanthomonas gardneri |
| 611 | Chateau Leoville, 2002 | 86040 | Xanthomonas citri |
| 130 | Merlot | 29760 | Vitis vinifera |
| 2057 | Primitivo del Salento, 2015 | 75379 | Thiomonas intermedia |
| 402 | Primitivo del Salento, 2015 | 292415 | Thiobacillus |
| | | | denitrificans |
| 249 | Primitivo del Salento, 2015 | 1972068 | Sulfurivermis fontis |
| 500 | Merlot | 796027 | Sugiyamaella |
| | | | lignohabitans |
| 358 | Rioja Crianza, 2015 | 47763 | Streptomyces lydicus |
| 462 | Rioja Crianza, 2015 | 1673076 | Sphingobium |
| | | | hydrophobicum |
| 363 | Rioja Crianza, 2015 | 135719 | Sphingobium amiense |
| 110 | Primitivo del Salento, 2015 | 4558 | Sorghum bicolor |
| 174 | Chateau Leoville, 2002 | 28526 | Solanum pennellii |
| 279 | Rioja Crianza, 2015 | 37927 | Sinomonas atrocyanea |
| 1851 | Rioja Crianza, 2015 | 1080349 | Saccharomyces |
| | | | eubayanus |
| 569 | Primitivo del Salento, 2015 | 43675 | Rothia mucilaginosa |
| 620 | Primitivo del Salento, 2015 | 762570 | Rhodothermus marinus |
| 600 | Merlot | 316056 | Rhodopseudomonas |
| | | | palustris |
| 565 | Chateau Leoville, 2002 | 206389 | Rhodocyclales |
| 164 | Primitivo del Salento, 2015 | 1457195 | Ralstonia |
| | | | solanacearum |
| 179 | Primitivo del Salento, 2015 | 104087 | Pseudomonas |
| | | | frederiksbergensis |
| 1400 | Merlot | 746360 | Pseudomonas |
| | | | fluorescens WH6 |
| 379 | Rioja Crianza, 2015 | 651740 | Pseudomonas cedrina |
| 1556 | Chateau Leoville, 2002 | 930166 | Pseudomonas |
| | | | brassicacearum |
| 2432 | Chateau Leoville, 2002 | 1249552 | Pseudohongiella |
| | | | spirulinae |
| 600 | Merlot | 2315862 | Pseudoflavitalea sp. |
| | | | 5GH32-13 |
| 915 | Chateau Leoville, 2002 | 43657 | Pseudoalteromonas |
| | | | luteoviolacea |
| 2000 | Merlot | 1774273 | Polaribacter vadi |
| 4000 | Merlot | 1416914 | Pandoraea pnomenusa |
| 248 | Rioja Crianza, 2015 | 93219 | Pandoraea |
| | | | norimbergensis |
| 238 | Rioja Crianza, 2015 | 323098 | Nitrobacter |
| | | | winogradskyi |
| 700 | Merlot | 700598 | Niastella koreensis |
| 3464 | Chateau Leoville, 2002 | 929713 | Niabella soli |
| 3000 | Merlot | 929704 | Myroides odoratus |
| | | | DSM 2801 |
| 906 | Chateau Leoville, 2002 | 1764 | Mycobacterium avium |
| 11860 | Chateau Leoville, 2002 | 714943 | Mucilaginibacter |
| | | | paludis |
| 267 | Rioja Crianza, 2015 | 1072463 | Microbacterium |
| | | | lemovicicum |
| 922 | Rioja Crianza, 2015 | 857087 | Methylomonas |
| | | | methanica |
| 231 | Rioja Crianza, 2015 | 395965 | Methylocella silvestris |
| 600 | Merlot | 265072 | Methylobacillus |
| | | | flagellatus |
| 116 | Primitivo del Salento, 2015 | 420662 | Methylibium |
| | | | petroleiphllum |
| 207 | Primitivo del Salento, 2015 | 3750 | Malus domestica |
| 861 | Chateau Leoville, 2002 | 186826 | Lactobacillacae |
| 113 | Primitivo del Salento, 2015 | 1349767 | Janthinobacterium |
| | | | agaricidamnosum |
| 895 | Chateau Leoville, 2002 | 1227739 | Hymenobacter |
| | | | swuensis |
| 388 | Rioja Crianza, 2015 | 1262470 | Herbaspirillum hiltneri |
| 500 | Merlot | 546367 | Hafnia alvei |
| 1315 | Chateau Leoville, 2002 | 426428 | Fusarium oxysporum |
| 1600 | Merlot | 459526 | Flavobacterium |
| | | | anhuiense |
| 2035 | Chateau Leoville, 2002 | 292691 | Flavobacter gramella |
| 4000 | Merlot | 1492898 | Flavisolibacter |
| | | | tropicus |
| 280 | Rioja Crianza, 2015 | 1257021 | Flammeovirgaceae |
| | | | bacterium |
| 6149 | Chateau Leoville, 2002 | 477680 | Filimonas lacunae |
| 2500 | Merlot | 471854 | Dyadobacter |
| | | | fermentans DSM |
| | | | 18053 |
| 200 | Primitivo del Salento, 2015 | 398580 | Dinoroseobacter |
| | | | shibae |
| 2347 | Chateau Leoville, 2002 | 985 | Cytophaga |
| | | | hutchinsonii |
| 1366 | Chateau Leoville, 2002 | 320787 | Cyclobacterium |
| | | | amurskyense |
| 600 | Merlot | 164546 | Cupriavidus |
| | | | taiwanensis |
| 355 | Rioja Crianza, 2015 | 381666 | Cupriavidus necator |
| 137 | Primitivo del Salento, 2015 | 1224164 | Corynebacterium |
| | | | vitaeruminis |
| 421 | Rioja Crianza, 2015 | 1144275 | Corallococcus |
| | | | coralloldes |
| 274 | Rioja Crianza, 2015 | 469383 | Conexibacter woesei |
| 8191 | Chateau Leoville, 2002 | 1492 | Clostridium butyricum |
| 3062 | Chateau Leoville, 2002 | 2321403 | Chryseolinea sp. |
| | | | KIS68-18 |
| 700 | Merlot | 878220 | Chryseobacterium sp. |
| | | | StRB126 |
| 2184 | Chateau Leoville, 2002 | 254 | Chryseobacterium |
| | | | indoltheticum |
| 800 | Merlot | 651561 | Chryseobacterium |
| | | | arthrosphaerae |
| 650 | Merlot | 485918 | Chitinophaga pinensis |
| 4113 | Chateau Leoville, 2002 | 688270 | Cellulophaga algicola |
| 4000 | Merlot | 1019 | Capnocytophaga |
| | | | sputigena |
| 672 | Primitivo del Salento, 2015 | 1136231 | Candida orthopsilosis |
| 238 | Rioja Crianza, 2015 | 1136497 | Brevibacterium |
| | | | siliguriense |
| 800 | Merlot | 463024 | Bordetella genomo |
| | | | sp. 6 |
| 914 | Chateau Leoville, 2002 | 866536 | Belliella baltica |
| 4183 | Primitivo del Salento, 2015 | 288004 | Beggiatoa |
| | | | leptomitoformis |
| 113 | Primitivo del Salento, 2015 | 1226968 | Azospirillum |
| | | | humicireducens |
| 323 | Primitivo del Salento, 2015 | 41977 | Azoarcus communis |
| 129 | Primitivo del Salento, 2015 | 510516 | Aspergillus oryzae |
| 3507 | Chateau Leoville, 2002 | 330879 | Aspergillus fumigatus |
| 6487 | Chateau Leoville, 2002 | 2341117 | Arachidococcus sp. |
| | | | KIS59-12 |
| 900 | Merlot | 1850526 | Arachidicoccus sp. |
| | | | BS20 |
| 821 | Chateau Leoville, 2002 | 889453 | Alkalitalea |
| | | | saponilacus |
| 261 | Rioja Crianza, 2015 | 178339 | Actinomyces |
| | | | hongkongensis |
| 4586 | Chateau Leoville, 2002 | 1324350 | Acinetobacter equi |
| 479 | Rioja Crianza, 2015 | 481146 | Acetobacter ascendens |
| 5011 | Chateau Leoville, 2002 | 746697 | Aequorivita |
| | | | sublithincola |
| 2902 | Chateau Leoville, 2002 | 160791 | Sphingomonas |
| | | | wittichii |
| 1557 | Chateau Leoville, 2002 | 1315974 | Sphingobium sp. |
| | | | TKS |
| 1711 | Chateau Leoville, 2002 | 135719 | Sphingobium amiense |
| 1619 | Chateau Leoville, 2002 | 1850238 | Rhizorhabdus |
| | | | dicambivorans |
| 1391 | All | 1122214 | Martelella |
| | | | mediterranea |
| | | | DSM 17316 |
| 1522 | All | 86040 | Xanthomonas citri pv. |
| | | | malvacearum |
| 1031 | All | 1176587 | Niabella |
| | | | ginsenosidivorans |
| 1112 | Chateau Leoville, 2002 | 880070 | Cyclobacterium |
| | | | marinum DSM 745 |

1. A method for evaluating the authenticity of a food product, the method comprising the steps of
a) obtaining a sample of the food product;
b) generating a plurality of signals based on the presence and/or the amount of two or more target molecules in the sample obtained in step (a), wherein the generation of the plurality of signals comprises a sequencing method and/or a microarray assay;
c) compiling an identifier having a plurality of elements based on the plurality of signals generated in step (b);
d) determining one or more properties the identifier of the food product is expected to have to be authentic;
e) comparing the one or more properties determined in step (d) for the food product to the respective one or more properties of an identifier for a product that is known to be authentic; and
f) evaluating the authenticity of the candidate product based on the comparison made in step (e).

2. (canceled)

3. The method according to claim 1, wherein the identifier for the product that is known to be authentic is compiled simultaneously with the identifier for the food product or wherein the identifier for the product that is known to be authentic is part of a library of identifiers.

4-8. (canceled)

9. The method according to claim 1, wherein the food product is a liquid processed food product selected from the group consisting of: olive oil, wine, whiskey and cognac.

10. The method according to claim 9, wherein the liquid is wine.

11. (canceled)

12. The method according to claim 1, wherein the two or more target molecules are comprised in the microbiome and/or the macrobiome of the food product and/or the product known to be authentic.

13. (canceled)

14. The method according to claim 12, wherein the two or more target molecules are bacterial nucleic acid molecules.

15. (canceled)

16. (canceled)

17. The method according to claim 14, wherein the nucleic acid molecules encode bacterial 16S ribosomal RNAs.

18-22. (canceled)

23. The method according to claim 1, wherein the microarray assay and/or the sequencing method comprises a hybridization step, wherein at least two nucleic acid molecules from the set of molecules capable of recognizing and/or binding selected target molecules hybridize with target nucleic acid molecules and/or nucleic acid molecules that have been obtained from target nucleic acid molecules.

24. The method according to claim 23, wherein the at least two nucleic acid molecules hybridize with nucleic acid molecules encoding a 16S ribosomal RNA and/or with nucleic acid molecules that have been obtained from nucleic acid molecules encoding a 16S ribosomal RNA.

25. (canceled)

26. The method according to claim 1, wherein compiling the identifier comprises a step of comparing the strengths of the plurality of signals generated in step (b) or generated by one or more additional analytical methods to one or more thresholds.

27. The method according to claim 26, wherein a target molecule is determined to be present in the sample, if the strength of the signal for said target molecule is above a threshold, or wherein a target molecule is determined to be absent in the sample, if the strength of the signal for said target molecule is below a threshold.

28-30. (canceled)

31. The method according to claim 26, wherein the one or more thresholds are defined based on the number of reads obtained by next generation sequencing.

32. The method according to claim 31, wherein a target molecule is determined to be comprised in a sample if at least 10 reads that correspond to said target molecule are identified in the sample by next generation sequencing.

33-40. (canceled)

41. The method according to claim 1, wherein compiling the identifier having a plurality of elements based on the plurality of signals comprises the steps of
a) determining at least one ratio of signal strengths; and
b) evaluating said ratio in view of at least one other ratio obtained for a different combination of signal strengths.

42. (canceled)

43. The method according to claim 41, wherein compiling the identifier comprises the generation of a binary matrix that comprises N bits with N corresponding to or being larger than the number of distinguishable different target molecules in the sample.

44. The method according to claim 1, wherein the food product is determined not to be authentic if one or more properties of the identifier of the food product do not compare favorably to the one or more corresponding properties of the identifier of the product that is known to be authentic.

45-47. (canceled)

48. The method according to claim 44, wherein rejecting the assumption that a food product is authentic is attempted in an iterative manner.

49. (canceled)

50. (canceled)

51. The method according to claim 44, wherein the evaluation of authenticity is complemented by one or more additional analytical methods.

52-60. (canceled)

61. A method for establishing a library of information for the authentication of a candidate product, the method comprising the steps of:
a) obtaining one or more samples from a plurality of genuine products; and
b) providing an identifier for each sample which comprises generating a plurality of signals based on the presence and/or the amount of two or more target molecules in the sample obtained in step (a), wherein the generation of the plurality of signals comprises a sequencing method and/or a microarray assay; and compiling an identifier having a plurality of elements based on the plurality of generated signals; and
c) establishing a library based on the information obtained in step (b).

62. The method according to claim 61, wherein establishing the library of information comprises defining a plurality of thresholds of signal strengths.

63-66. (canceled)


SEQUENCE LISTING (23 sequences)

SEQ ID NO: 1 - DNA, 415 bp, Acetobacter sp.
  misc_feature: Bacteria, prokaryote
cctacgggtggctgcagtggggaattttggacaatgggcgaaagcctgatccagcaatgccgcgtgtgtgagagtaactgctcatgcagtgacggtatccaaccagaaagccccggctaacttcgtgccagcagccgcggtaatacgaagggggctagcgttgctcggaatgactgggcgtaaagggcgtgtaggcggtttgtacagtcagatgtgaaatccccgggcttaacctgggagctgcatttgatacgtgcagactagagtgtgagagagggttgtggaattcccagtgtagaggtgaaattcgtagatattcggaagaacaccagtggcgaaggcgactcgctggacaagtattgacgctgaggtgcgaaagcgtggggagcaaacaggattagataccccggtagtc

SEQ ID NO: 2 - DNA, 465 bp, Acinetobacter sp.
  misc_feature: Bacteria, prokaryote
cctacgggaggcagcagtggggaatattggacaatggggggaaccctgatccagccatgccgcgtgtgtgaagaaggccttttggttgtaaagcactttaagttgggaggaagggcattaacctaatacgttagtgttttgacgttaccgacagaataagcaccggctaactctgtgccagcagccgcggtaatacagagggtgcaagcgttaatcggatttactgggcgtaaagcgcgcgtaggtggccaattaagtcaaatgtgaaatccccgagcttaacttgggaattgcattcgatactggttggctagagtatgggagaggatggtagaattccaggtgtagcggtgaaatgcgtagagatctggaggaataccgatggcgaaggcagccatctggcctaatactgacactgaggtgcgaaagcatggggagcaaacaggattagatacccgagtagtc

SEQ ID NO: 3 - DNA, 465 bp, Bacillus sp.
  misc_feature: Bacteria, prokaryote
cctacgggaggcagcagtagggaatcttccgcaatggacgaaagtctgacggagcaacgtcgcgtgagtgatgaaggctttcgggtcgtaaaactctgttgttagggaagaacaagtgctagttgaataagctggcaccttgacggtacctaaccagaaagccacggctaactacgtgccagcagccgcggtaatacagagggtgcgagcgttaatcggatttactgggcgtaaagcgcgcgcaggcggctatgtaagtctggtgttaaagcccggagctcaactccggttcgcatcggaaactgtgtagcttgagtgcagaagaggaaagcggtattccacgtgtagcggtgaaatgcgtagagatgtggaggaacaccagtggcgaaggcggctttctggtctgtaactgacactgaggcgcgaaagcgtggggagcaaacaggattagatacccgagtagtc

SEQ ID NO: 4 - DNA, 465 bp, Brevibacillus sp.
  misc_feature: Bacteria, prokaryote
cctacggggggctgcagtagggaattttccacaatggacgaaagtctgatggagcaacgccgcgtgaacgatgaaggtcttcggattgtaaagttctgttgtcagggatgaacacgtaccgttcgaatagggcggtaccttgacggtacctgacgagaaagccacggctaactacgtgccagcagccgcggtaatacgtaggtggcaagcgttgtccggatttattgggcgtaaagcgcgcgcaggcggctatgtaagtctggtgttaaagcccggagctcaactccggttcgcatcggaaactgtgtagcttgagtctcgtagaggggggtagaattccaggtgtagcggtgaaatgcgtagagatctggaggaataccggtggcgaaggcggccccctggacgaagactgacgctcaggtgcgaaagcgtggggagcaaacaggattagataccccagtagtc

SEQ ID NO: 5 - DNA, 465 bp, Burkholderia sp.
  misc_feature: Bacteria, prokaryote
cctacggggggcagcagtggggaatattggacaatgggcgcaagcctgatccagcaatgccgcgtgtgtgaagaaggtcttcggattgtaaagcgcttttgtccgggaagaaatcctttctgataataccggagggggatgacggtaccggaagaagaagccccggctaacttcgtgccagcagccgcggtaatacgtagggtgcgagcgttaatcggaattactgggcgtaaagcgtgcgcaggcggtgatgtaagaccgatgtgaaatccccgggcttaacctgggaactgcattggtgactgcatcgctggagtatggcagaggggggtggaattccacgtgtagcagtgaaatgcgtagagatgtggaggaacaccgatggcgaaggcagccccctgggccaatactgacgctcatgcacgaaagcgtggggagcaaacaggattagatacccgggtagtc

SEQ ID NO: 6 - DNA, 440 bp, Dyella sp.
  misc_feature: Bacteria, prokaryote
cctacgggaggctgcagtggggaatattggacaatgggcgcaagcctgatccagcaatgccgcgtgtgtgaagaaggtcttcggattgtaaagcactttcgacggggacgatgatgacggtacccgtagaagaagccccggctaacttcgtgccagcagccgcggtaatacgaagggggctagcgttgctcggaatgactgggcgtaaagcgtgcgcaggcggtgatgtaagaccgatgcgaaatccccgggcttaacctgggaatggcagtggatactggatcgctagagtgtgatagaggatggtggaattcccggtgtagcggtgaaatgcgtagagatcgggaggaacatcagtggcgaaggcggccatctggatcaacactgacgctgaggcacgaaagcgtggggagcaaacaggattagataccccagtagtc

SEQ ID NO: 7 - DNA, 466 bp, Oenococcus sp.
  misc_feature: Bacteria, prokaryote
cctacgggcggcagcagtggggaatattggacaatgggcgcaagcctgatccagcaatgccgcgtgtgtgatgaaggctttcgggtcgtaaagcactgttgtaagggaagaataactgaattcagagaaagttttcagcttgacggtaccttaccagaaagggatggctaaatacgtgccagcagccgcggtaatacgtatgtcccgagcgttatccggatttattgggcgtaaagcgagcgcagacggtttattaagtctgatgtgaaatcccgaggcccaacctcggaactgcattggaaactgatttacttgagtgcgatagaggcaagtggaactccatgtgtagcggtgaaatgcgtagatatctggaagaacaccgatggcgaaggcaacctcctgggcctgttctgacgctgaggcacgaaagcgtgggtagcaaacaggattagataccccagtagtc

SEQ ID NO: 8 - DNA, 465 bp, Pelomonas sp.
  misc_feature: Bacteria, prokaryote
cctacgggtggctgcagtggggaattttggacaatgggcgcaagcctgatccagccatgccgcgtgcgggaagaaggccttcgggttgtaaaccgcttttgtcagggaagaaaaggttctggttaatacctgggactcatgacggtacctgaagaataagcaccggctaactccgtgccagcagccgcggtaatacggagggtgcaagcgttaatcggaattactgggcgtaaagcgcacgcaggcggtttgttaagtcagatgtgaaatccccgggctcaacctgggaactgcatctgatactggcaagcttgagtctcgtagaggggggtagaattccaggtgtagcggtgaaatgcgtagagatctggaggaataccgatggcgaaggcaggttactgggcagttactgacgctgaggagcgaaagcatgggtagcaaacaggattagataccctggtagtc

SEQ ID NO: 9 - DNA, 465 bp, Salinispora sp.
  misc_feature: Bacteria, prokaryote
cctacggggggctgcagtggggaattttggacaatgggggaaaccctgatccagcaatgccgcgtgtgtgaagaaggccttcgggttgtaaagcacttttgtccgggaagaaatcctttctgataatactggagggggatgacggtaccggaagaataagcaccggctaactacgtgccagcagccgcggtaatacgcagggtgcgagcgttaatcggaattactgggcgtaaagcgtgcgcaggcggtgatgtaagaccgatgtgaaatccccgggcttaacctgggaactgcattggtgactgcattgctggagtatggcagaggggggtggaattccacgtgtagcagtgaaatgcgtagagatgtggaggaacaccgatggcgaaggcagccccctgggccaatactgacgctcatgcacgaaagcgtggggagcaaacaggattagatacccgtgtaatc

SEQ ID NO: 10 - DNA, 465 bp, Streptococcus sp.
  misc_feature: Bacteria, prokaryote
cctacggggggctgcagtagggaatcttcggcaatggggggaaccctgaccgagcaacgccgcgtgagtgaagaaggttttcggatcgtaaagctctgttgtaagagaagaacgggtgtgagagtggaaagttcacactgtgacggtatcttaccagaaagggacggctaactacgtgccagcagccgcggtaatacgtaggtcccgagcgttgtccggatttattgggcgtaaagcgagcgcaggcggttagataagtctgaagttaaaggctgtggcttaaccatagtatgctttggaaactgtttaacttgagtgcagaaggggagagtggaattccatgtgtagcggtgaaatgcgtagatatatggaggaacaccagtggcgaaggcgactttctggtctgtaactgacactgaggcgcgaaagcgtggggagcaaacaggattagataccctggtagtc

SEQ ID NO: 11 - DNA, 50 bp, Artificial Sequence
  Synthetic primer V3
  misc_feature (42)..(42): n is a, c, g, or t
tcgtcggcagcgtcagatgtgtataagagacagcctacgggnggcwgcag

SEQ ID NO: 12 - DNA, 55 bp, Artificial Sequence
  Synthetic primer V4
gtctcgtgggctcggagatgtgtataagagacaggactachvgggtatctaatcc

SEQ ID NO: 13 - DNA, 51 bp, Artificial Sequence
  Synthetic primer NGS-i5-S518
aatgatacggcgaccaccgagatctacacctattaagtcgtcggcagcgtc

SEQ ID NO: 14 - DNA, 51 bp, Artificial Sequence; identical to SEQ ID NO: 13
  Synthetic primer NGS-i5-S520

SEQ ID NO: 15 - DNA, 51 bp, Artificial Sequence
  Synthetic primer NGS-i5-S521
aatgatacggcgaccaccgagatctacacgagccttatcgtcggcagcgtc

SEQ ID NO: 16 - DNA, 47 bp, Artificial Sequence
  Synthetic primer NGS-i5-N720
caagcagaagacggcatacgagataggctccggtctcgtgggctcgg

SEQ ID NO: 17 - DNA, 47 bp, Artificial Sequence
  Synthetic primer NGS-i5-N721
caagcagaagacggcatacgagatgcagcgtagtctcgtgggctcgg

SEQ ID NO: 18 - DNA, 47 bp, Artificial Sequence
  Synthetic primer NGS-i5-N722
caagcagaagacggcatacgagatctgcgcatgtctcgtgggctcgg

SEQ ID NO: 19 - DNA, 47 bp, Artificial Sequence
  Synthetic primer NGS-i5-N723
caagcagaagacggcatacgagatgagcgctagtctcgtgggctcgg

SEQ ID NO: 20 - DNA, 47 bp, Artificial Sequence
  Synthetic primer NGS-i5-N724
caagcagaagacggcatacgagatcgctcagtgtctcgtgggctcgg

SEQ ID NO: 21 - DNA, 47 bp, Artificial Sequence
  Synthetic primer NGS-i5-N726
caagcagaagacggcatacgagatgtcttagggtctcgtgggctcgg

SEQ ID NO: 22 - DNA, 47 bp, Artificial Sequence
  Synthetic primer NGS-i5-N727
caagcagaagacggcatacgagatactgatcggtctcgtgggctcgg

SEQ ID NO: 23 - DNA, 47 bp, Artificial Sequence
  Synthetic primer NGS-i5-N728
caagcagaagacggcatacgagattagctgcagtctcgtgggctcgg